(19) United States
(12) Patent Application Publication
(10) Pub. No.: US 2024/0188833 A1
(43) Pub. Date: Jun. 13, 2024

(54) SYSTEMS, APPARATUS AND METHODS FOR DETERMINING ANEURYSM AND ARTERIAL WALL ENHANCEMENT

(71) Applicant: University of lowa Research Foundation, Iowa City, IA (US)

(72) Inventors: Edgar Samaneigo, Iowa city, IA (US); Tim Koscik, Iowa City, IA (US)

(21) Appl. No.: 18/555,381

(22) PCT Filed: Apr. 13, 2022
(86) PCT No.: PCT/US2022/024707

§ 371 Date: Oct. 13, 2023

Related U.S. Application Data
(60) Provisional application No. 63/174,483, filed on Apr. 13, 2021.

(51) Int. Cl. A61B 5/02; A61B 5/055; G06T 7/00; G06T 7/62; G16H 50/30

(52) U.S. Cl. CPC A61B 5/02014; A61B 5/055; G06T 7/0014; G06T 7/62; G16H 50/30; A61B 2576/02; G06T 2207/10088; G06T 2207/20036; G06T 2207/30016; G06T 2207/30101

(57) ABSTRACT

The disclosed apparatus, systems and methods relate to devices, systems and methods for measuring aneurysm wall enhancement and intracranial wall enhancement.

### CROSS-REFERENCE TO RELATED APPLICATION(S)

This application claims the benefit under 35 U.S.C. § 119(e) to U.S. Provisional Application 63/174,483, filed Apr. 13, 2021, and entitled “Systems, Apparatus and Methods for Determining Aneurysm Wall Enhancement,” which is hereby incorporated herein by reference in its entirety for all purposes.

### TECHNICAL FIELD

The disclosed technology relates generally to detection of tissue characteristics, and in particular, to the devices, methods, and design principles allowing the user to identify the characteristics of individual tissues such as aneurysm tissues and arterial walls. This has implications for research, diagnosis, and treatment.

### BACKGROUND

The disclosure relates to apparatus, systems and methods for improved techniques for establishing the characteristics of tissues such as damaged tissues.

High resolution vessel wall imaging techniques with, for example, 7T-MRI and 3T-MRI provide insight of possible biological processes that determine aneurysm wall enhancement (AWE) and affect the wall. These biological processes may ultimately lead aneurysm growth and rupture.

High resolution-magnetic resonance imaging (HR-MRI) provides a high contrast-to-noise ratio and spatial resolution for better visualization of intracranial and other arterial walls. Biological processes such as microhemorrhages, wall thickening, atherosclerotic changes or indirect signs of inflammation may be visualized with HR-MRI. Within this spectrum of radiological findings, aneurysmal wall enhancement (AWE) after the administration of gadolinium (Gd) has been described as a marker of aneurysm instability. The characterization and understanding of AWE as a surrogate marker of changes in the aneurysm wall biology is a nascent field.

Histological analysis of aneurysm walls show endothelial disruption as a potential marker aneurysm instability. Formation of atherosclerotic lesions as a result of lipid accumulation in the aneurysm wall is one example of the complex remodeling mechanisms found in aneurysm walls (FIG. 1). Histological analysis has linked gadolinium uptake in the aneurysm wall to intimal disruptions in the internal elastic lamina and endothelial cells. Understanding how dynamic contrast uptake at different layers of the aneurysm wall (as shown in FIG. 5) can impact AWE is essential to characterizing AWE as a biomarker for instability.

Meta-analysis and several reports have consistently shown that AWE is significantly correlated with higher risk of aneurysm growth and rupture. The definitions of AWE vary broadly, although recently there has been a step forward in determining AWE objectively instead of subjectively. One of the major limitations in the current method of assessing AWE is that images are processed and analyzed in multiplanar 2D views and in the best-case scenario, include different segments of the aneurysm. This approach is limited by the manual sampling performed by the investigator in determining which areas of the aneurysm have increased AWE.

There is also no consensus in determining which areas of the aneurysm wall should be analyzed. There is need in the art for such technologies.

### BRIEF SUMMARY

Discussed herein are various devices, systems and methods relating to the analysis of tissue regions, such as aneurysm walls. In various implementations, these methods and devices are used in an AWE system to generate objective AWE measurements for use in research and clinical applications. The method developed can be applied in the analysis of any arterial wall, which may include but is not limited to cerebral arteries, coronary arteries, and the aorta.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes an aneurysm wall enhancement (AWE) measurement method. The aneurysm wall enhancement also includes retrieving scan data, processing scan data, and modeling processed scans to generate an aneurysm surface model and/or histograms. Other embodiments of this aspect include corresponding computer systems, apparatus, and computer programs recorded on one or more computer storage devices, each configured to perform the actions of the methods.

In Example 1, a method for analyzing an arterial wall comprising: obtaining a scan of a target area; processing the scan; analyzing surface of the target area; calculating one or more metrics for comparison; and outputting an instability outcome.

Example 2 relates to the method of Example 1, wherein the one or more metrics include one or more of diameter, size ratio, aspect ratio, ellipticity index, non-sphericity index, undulation index, surface area, volume, wall thickness, bleb percentage, and mural thrombosis.

Example 3 relates to the method of Example 1 or 2, wherein the instability outcome is one or more of a likelihood of stroke, indication of aneurysm size, likelihood of rupture, or indication of inflammation of an artery.

Example 4 relates to the method of any of Examples 1-3, further comprising aligning the scan to a template.

Example 5 relates to the method of any of Examples 1-4, further comprising extracting spoke variables.

Example 6 relates to the method of any of Examples 1-5, further comprising extracting surface measures.

Example 7 relates to the method of any of Examples 1-6, further comprising measuring gadolinium uptake.

Example 8 relates to the method of any of Examples 1-7, further comprising determining one or more of aneurysm wall enhancement, generalized aneurysm wall enhancement, specific aneurysm wall enhancement, circumferential aneurysm wall enhancement, and focal aneurysm wall enhancement.

Example 9 relates to the method of any of Examples 1-8, further comprising projecting spokes into the target area.

Example 10 relates to the method of Example 9, further comprising probing a signal intensity along each spoke.

Example 11 relates to the method of Example 10, further comprising store a maximum signal intensity for each spoke.

Example 12 relates to the method of any of Examples 9-11, further comprising normalizing spoke values to a reference structure.

Example 13 relates to the method of Example 12, wherein the reference structure is a pituitary stalk or a corpus callosum.

Example 14 relates to the method of Example 13, wherein the reference structure is the corpus callosum.

Example 15 relates to the method of any of Examples 1-14, further comprising calculating morphological indices.

Example 16 relates to the method of any of Examples 1-15, wherein gadolinium is administered prior to the scan.

Example 17 relates to the method of any of Examples 1-16, further comprising determining a contrast ratio.

Example 18 relates to the method of Example 17, wherein the contrast ratio is normalized to a reference structure.

Example 19 relates to the method of any of Examples 1-18, further comprising comparing the one or more metrics for two scans, one taken before administration of contrast, and one taken after administration of contrast.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows vessel wall layers of an intracranial aneurysm with a bleb (arrow) and atherosclerotic calcification (arrowhead). The aneurysm wall has a complex morphology with multiple biological processes. The aneurysm represents an abnormal “outpouching” of the parent artery.

FIG. 2A is a flow chart showing various optional steps and sub-steps performed by the system, according to certain exemplary implementations.

FIG. 2B is a further flow chart showing further implementations of the optional steps and sub-steps utilized by the system in certain implementations.

FIG. 2C is a system diagram of the system, according to one implementation.

FIG. 3 shows 3D heat maps of T1-weighted (T1) and T1+Gd AWE of a post inferior cerebellar artery aneurysm. Histograms were generated from 3D maps: y axis=spoke density and x axis=AWE raw values (first row) and AWE ratios for the pituitary stalk (second row) and corpus callosum (third row). The corpus callosum was a better structure for normalization and had an almost identical curve when compared to raw values (first and third rows). Avid pituitary stalk Gd enhancement shifts the ratio to the left in T1+Gd images (middle row). This aneurysm is highly enhancing as show in T1+Gd images.

FIG. 4 shows a comparison of 2D planar views on T1+Gd images (left column), semi-automated 3D maps of AWE from T1+Gd images (center) and histograms. 3D heat maps provide a comprehensive topographic view of AWE. Histogram analysis of each aneurysm (right column) quantifies dynamic changes in AWE before and after contrast administration: specific to each aneurysm (SAWE), against generalized thresholds for enhancement (GAWE) and identifies regions of focal AWE (FAWE). Panel A shows an M2 aneurysm with a daughter sac (red arrowhead) and areas of FAWE identified as the area under the right tail (red) on the histogram. The mean T1+Gd values do not meet the established thresholds for SAWE (>two SDs from the mean T1=black dotted line) or GAWE (CC ratio≥1=blue arrow). Although the aneurysm appears enhancing on 2D views (arrowhead), AWE analysis by SAWE and GAWE does not demonstrate enhancement. The pseudo-AWE seen on 2D may be related to slow flow in this distal M2 bifurcation aneurysm. Panel B shows a SAWE+/GAWE− basilar tip aneurysm. The aneurysm does not exhibit FAWE. On 2D view, the aneurysm does not appear enhancing, despite its known high risk of rupture: basilar location. Panel C shows a highly enhancing post inferior cerebellar artery aneurysm (arrowhead) with documented growth over time. Histogram analysis shows that this aneurysm is both SAWE+ (black arrow) and GAWE+ (blue arrow). FAWE was visualized in the histogram and 3D heat map. This aneurysm has benign morphological features that make it a “low risk of rupture” (size=3.8 mm, AR=1.76). Panel D shows a highly enhancing internal carotid artery aneurysm on T1+Gd imaging (left). On histogram analysis this aneurysm was GAWE+(blue arrow) and SAWE−. Additionally, the area of FAWE identified on the histogram (red) is visualized on the left sidewall of the aneurysm (arrowhead) on the 3D heatmap. 3D heat maps and histograms were generated on aneurysm specific scales to better visualize the enhancement topography. Manual and semi-automated CC ratios were very similar for all aneurysms.

FIG. 5 shows high-resolution images generated from 7T-MRI and processed to optimize co-registration, decrease artifact and facilitate masking. The 3D rotational angiogram demonstrates an anterior communicating artery aneurysm with two blebs (arrowheads). Spokes are projected from the inner lumen to the outer boundary of the aneurysm wall and are used to generate a 3D heat map (center row). The length of each spoke is tailored to the thickness of the aneurysm wall. AWE is normalized to the CC in T1 and T1+Gd images and is used to generate the histogram that matches the 3D heat map on the same scale. Blebs were isolated (bottom left) and AWE values were compared against manual regions of interest drawn on 2D images (bottom right). There was a high correlation between values generated by the semi-automated and manual methods. Blebs, otherwise not seen on 2D multiplanar imaging, were visualized and analyzed on 3D heat maps.

FIG. 6A shows Gd uptake of the aneurysm sac and its bleb (CC ratio≥1). Fourteen aneurysms had blebs, and 3 aneurysms had two blebs (1&2,6&7,16&17). Gd bleb uptake varied within the same aneurysm. Overall, blebs exhibited more enhancement than sacs.

FIG. 6B shows GAWE region of interest analysis (CC ratio≥1). Aspect ratio≥3.56 (specificity 95%, sensitivity 57%) and size ratio≥2.89 (specificity 76%, sensitivity 71%) best predicted GAWE+ aneurysms.

FIG. 7 schematic representation of the pipeline for generation of 3D AWE maps. Images are acquired with a high-resolution MRI protocol. Generation of 3D heatmaps of the aneurysm through spokes directed outward from the lumen of the aneurysm. Histograms can be generated from the 3D heatmaps for better visualization of AWE in T1 and T1+Gd images.

FIG. 8 shows a saccular GAWE−/SAWE+ anterior communicating artery aneurysm with significant Gd uptake demonstrated on T1 (Panel A) and T1+Gd (Panel B) heatmap reconstructions. Planar 7T-MRI views show minor changes in enhancement, which are not apparent in 2D views. Based on histogram analysis (Panel C), the aneurysm is GAWE− (μₚₒₛₜ<1) and SAWE+(μₚₒₛₜ≥μₚᵣₑ+2σ). The survival plot (Panel D) shows that ˜82% of spokes enhanced above the SAWE cutoff, and ˜2.5% of spokes enhanced above the FAWE cutoff. The circumferential SI of this aneurysm is μₚₒₛₜ=0.70. SAWE analysis determined that this aneurysm enhances, therefore is at higher risk of rupture (anterior communicating artery location, presence of multiple blebs, aspect ratio=1.68).

FIG. 9 shows a GAWE−/SAWE+ fusiform left internal carotid artery terminus aneurysm with no enhancement on T1 (Panel A) and heterogenous enhancement on T1+Gd (Panel B, arrowhead) reconstructions. Despite subjective enhancement from 7T-MRI, this aneurysm is considered GAWE−, because only 13% of spokes enhance more than the CC (Panel D). Approximately ˜71% of spokes (Panel D) enhance above the SAWE cutoff (CCᵣₐₜᵢₒ≥0.8), therefore this aneurysm is SAWE+. Areas of focal enhancement can be visualized on both the 3D reconstruction (arrow) and 7T-MRI (arrowhead) and are represented in the right tail of the histogram (Panel C). The survival plot (Panel D) shows that the area of focal enhancement comprises only ˜3% of the spokes in the aneurysm. This fusiform aneurysm uptakes Gd (SAWE+), but is not highly enhancing when compared with other aneurysms (GAWE−). Subjective analysis would have classified this aneurysm as enhancing (Panel B, inlet).

FIG. 10 shows a large thrombosed GAWE+/SAWE− vertebrobasilar aneurysm visualized on T1 (Panel A) and T1+Gd (Panel B) imaging. 7T-MRI shows that the inner wall has avid enhancement compared to the outer layers of the aneurysm sac that encompass the mural thrombus. Areas of focal enhancement on T1 reconstructions (Panel A) are not visualized on T1+Gd reconstructions (Panel B), where new areas of focal enhancement can be visualized. The irregular pattern of enhancement between T1 and T1+Gd is better visualized in the change of histogram shape (Panel C). The survival plot (Panel D) shows that both the T1 and T1+Gd spokes localize above the GAWE threshold (˜59% and ˜64% respectively). There is more focal enhancement T1 compared to T1+Gd reconstructions, as shown in the survival plot. This case illustrates the complex dynamics of Gd uptake in aneurysms with mural thrombosis.

FIG. 11 shows a 3D-AWE map of a post inferior cerebellar artery aneurysm. (A) Aneurysm segmentations are manually created with 3T HR-MRI, using T1 (top) and T1+Gd (bottom) images. (B) 3D-AWE mapping is generated from orthogonal probes that are automatically extended from the lumen into the aneurysm wall (center). The manual signal intensity (SI) values identified in T1 and T1+Gd images are similar to those obtained through 3D-AWE mapping (T1: 129 vs. 141 and T1+Gd: 329 vs. 339). (C) Quantification of AWE can be visualized through histograms (top) that show the distribution of SI throughout the aneurysm on T1 (black) and T1+Gd (red) images and survival plots (bottom) that identify the surface area of enhancement above different thresholds of SI. Mean SI increases from T1 to T1+Gd imaging with a right shift of the histogram (top). This is defined as SAWE (purple double arrow). There is significant positive skew of the histogram, which is defined as FAWE (top). The survival plot (bottom) shows that on the T1 image, 16% of the aneurysm surface has an SI greater than 141. On the T1+Gd image, only 2% of the aneurysm surface has an SI greater than 339. FAWE is highly concentrated in this area, suggesting that it may be prone to rupture (B, bottom).

FIG. 12 shows cerebrospinal fluid surrounding the aneurysm (A, arrow) creates an artificial gradient and skews down 3D-CAWE by creating bimodally distributed histograms (B). By filtering out values below the 70th percentile, this can be addressed to probe only the visible portion of the aneurysm wall (C).

FIG. 13 Manual measurements of CRₛₜₐₗₖ (contrast ratio normalized with the pituitary stalk) were strongly correlated to semiautomated measurements.

FIG. 14 ROC analysis predicting symptomatic status indicates that aneurysm size and circumferential aneurysm wall enhancement (CAWE) are strong predictors of symptomatic status.

FIG. 15 shows AWE maps of asymptomatic versus symptomatic aneurysms. (A) An asymptomatic 11-mm basilar tip aneurysm (top) shows a uniform distribution of AWE on the histogram (center) and barely uptakes Gd. This aneurysm is 3D-CAWE-(3D-CAWE<1). (B) Conversely, a symptomatic 3.5-mm anterior communicating artery aneurysm (bottom) is 3D-CAWE+ and has a significant positive skew (FAWE) on the histogram (center). The survival plot (right) shows that approximately 10% of the aneurysm has FAWE. Although this aneurysm is small, it is symptomatic and has a large area of FAWE.

FIG. 16 is a 3D AWE map of an anterior communicating artery aneurysm with a bleb. A 4.6-mm anterior communicating artery aneurysm with a bleb that has increased Gd uptake, from T1 (A) to T1+Gd (B) images. The Gd uptake of the bleb is 172% higher than the rest of the aneurysm. The histogram (C) shows a broad distribution of signal intensity (SI) along the entire aneurysm, while the survival plot (D) demonstrates that the percentage of the aneurysm that enhances more than the bleb decreases from T1 (59%) to T1+Gd (32%). This indicates that the bleb picks up more Gd than the rest of the aneurysm.

FIG. 17 is a 3D AWE map and quantitative sustainability mapping (QSM) reconstruction of a basilar tip aneurysm. A QSM+7.7-mm basilar tip aneurysm with colocalization of increased SAWE (purple) from T1 (A) to T1+Gd (B) and a microhemorrhage (C). The histogram (D) shows a large right tail (skew=0.35) due to FAWE.

FIG. 18 shows generation of 3D enhancement maps. (A) The SI sampled in the high-resolution image of 7T MRI is almost the same to the generated through 3D mapping: 1517 and 1502. (B) Probes (arrows) extend from the arterial lumen to the plaque and contiguous vessel wall. An average of 125 probes/data points are generated per plaque and 858 per arterial segment. This technique allows the generation of a detailed color map of Gd enhancement.

FIG. 19 shows a schematic representation of how probe length was determined. The plaque thickness is estimated using the diameter of the vessel minus the diameter at the most stenotic segment, which was then divided by two. In this way the probe did not extend beyond the plaque.

FIG. 20 shows 3D enhancement maps of culprit and non-culprit plaques. Top: (A). A left middle cerebral artery culprit plaque is seen in a 7T MRI coronal view (arrowhead). The 3D reconstruction color maps of this middle cerebral artery plaque in T1 (B) and T1+Gd sequences (C) show high concentration of Gd enhancement (arrow). (A). Bottom: A non-culprit area of enhancement is seen in the left posterior cerebral artery on 7T MRI (arrowhead). The 3D color map is depicted in T1 (E) and T1+Gd (F). On 7T MRI this arterial segment appears highly enhancing (arrowhead) (D). However, the 3D color map does not show avid enhancement (arrow). Moreover, this arterial segment has lower Gd-uptake and plaque vs arterial enhancement than the culprit plaque.

### DETAILED DESCRIPTION

The various embodiments disclosed or contemplated herein relate to various apparatus, systems, and methods for the analysis of certain tissue regions, such as aneurysm wall enhancement (AWE) analysis. While certain implementations are directed to use in the analysis of AWE, it is readily appreciated that the principles, technologies, and approaches are contemplated to be readily adaptable to use in other tissue types and for further purposes, as would be readily appreciated by those of skill in the art, including but not limited to arterial wall enhancement.

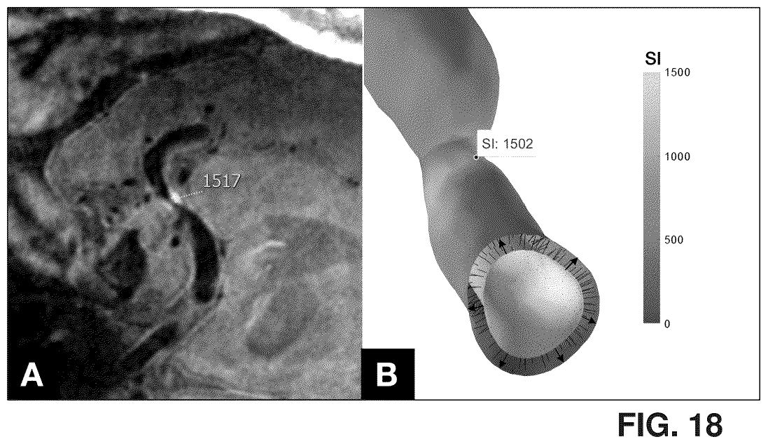

In this disclosure, the disclosed method and related systems of quantifying AWE with 3D maps and histogram analysis generates metrics that correlate with known morphological features of aneurysm and arterial wall instability. Compared to previous subjective 2D-multiplanar methods of defining enhancement, the disclosed approach provides reliable qualitative and quantitative metrics that can be used to measure aneurysm-specific topographical features and differences between different aneurysms. This method can also be used to analyze other arterial wall segments that may be affected by diseases such as: atherosclerosis, diabetes, hypertension, and congenital inherent wall defects. 3D maps and histogram analysis can be generated for any arterial wall through this method (see for example FIG. 18, discussed further below).

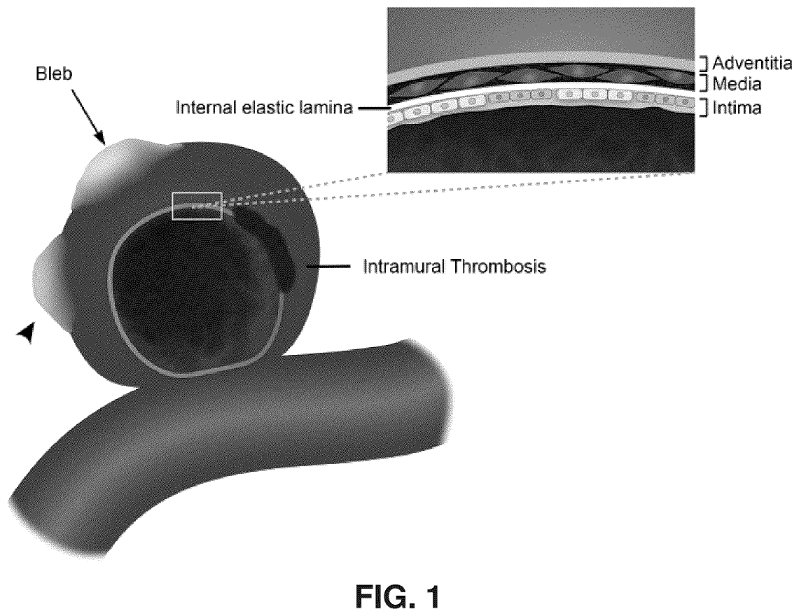

Detailed analysis of focal AWE (FAWE) has determined that the co-localization of these areas with low wall shear stress, such as blebs (FIG. 1) is associated with a higher risk of rupture. Other groups determined a higher risk of rupture when circumferential aneurysmal wall enhancement (CAWE) is present, instead of FAWE. Intramural thrombosis (FIG. 1) has also been associated with increased CAWE. These different approaches in analyzing AWE are aimed at identifying aneurysms prone to grow and rupture. However, these approaches have the inherent limitation of using manual 2D multiplanar sampling to characterize a 3D structure with complex morphology such as brain aneurysms (FIG. 1). Aneurysm size and morphological indices such as size and aspect ratios have been correlated with a higher risk of rupture. These indices may be used in validating new methods of AWE characterization.

Various embodiments discussed herein relate to a semi-automated method of generating 3D-AWE maps of the entire aneurysm. The main aim of this approach is to characterize different biological processes that affect the aneurysm wall and may manifest with different patterns of AWE. A comprehensive 3D analysis of the aneurysm will lead to a better estimation of the risk of rupture. Further the systems, methods, and associated analysis allow for screening and detection of aneurysms that may grow and/or rupture, aneurysms that are stable and may not require treatment, arterial segments that may cause strokes, and inflamed arteries. In certain implementations, the systems and methods allow for a determination of stroke risk.

Aneurysm wall enhancement (AWE) after the administration of contrast gadolinium (Gd) has been identified as a potential biomarker of unstable intercranial aneurysms. While most studies determine AWE subjectively on multiplanar-2D views, the disclosed systems and methods comprehensively quantified AWE in 3D using a semi-automated method. It is appreciated, and would be readily apparent to those of skill in the art, that any of field strength of MRI or imager can be utilized in alternate implementations, and that no mention of the field strength used here should be considered limiting.

The signal intensity (SI) of the aneurysm wall was mapped using orthogonal spokes tailored to the aneurysm wall thickness to maximize accuracy. SI values were normalized to the pituitary stalk and corpus callosum (CC) and compared to manual measurements to determine the accuracy of the new semi-automated method. Correlation between 3D and 2D standard measurements of AWE was high for both the CC and pituitary stalk ratios (r=0.792 vs r=0.806), but the variability in CC ratios vs pituitary stalk ratios (CV 28% vs 38%) proved the CC to be a more reliable normalizing structure. 3D heatmaps and histograms of AWE were used to generate the following AWE metrics: specific enhancement (SAWE), general enhancement (GAWE), and focal enhancement (FAWE). GAWE is useful in discerning differences in wall thickness, size, size ratio, and aspect ratio. SAWE and FAWE were aneurysm specific metrics used to identify contrast related enhancement and areas of focal enhancement in the aneurysm. 3D-AWE mapping may be a powerful objective tool in determining AWE and generating a new set of metrics to analyze biological processes of the aneurysm wall that may affect wall enhancement.

Various implementations of the disclosed systems and methods of quantifying AWE and arterial wall enhancement comprise certain hardware and software components configured for the execution of one or more steps and/or sub-steps. In certain of these implementations, a system of one or more computers can be configured to perform particular operations or actions by virtue of having software, firmware, hardware, or a combination of them installed on the system that in operation causes or cause the system to perform the actions. In exemplary embodiments of the system 10, a computer platform comprising a computer-readable media and a data processing system are provided for recording, monitoring and modifying the raw data and generating the visual images described herein in reference to the below examples.

As described herein in greater detail, each of the exemplary implementations of the disclosed system comprises several optional steps and, optionally, sub-steps which may be performed in any order. It is appreciated that while certain of the steps and/or sub-steps may naturally occur in a certain order, no specified order is necessarily provided for by the use of numbers or the order in which they are described herein. Further, it is readily appreciated that various steps are optional, or can be omitted, and that the provision of an exemplary implementation does not mean that each of the listed steps is required for the described systems and methods to function.

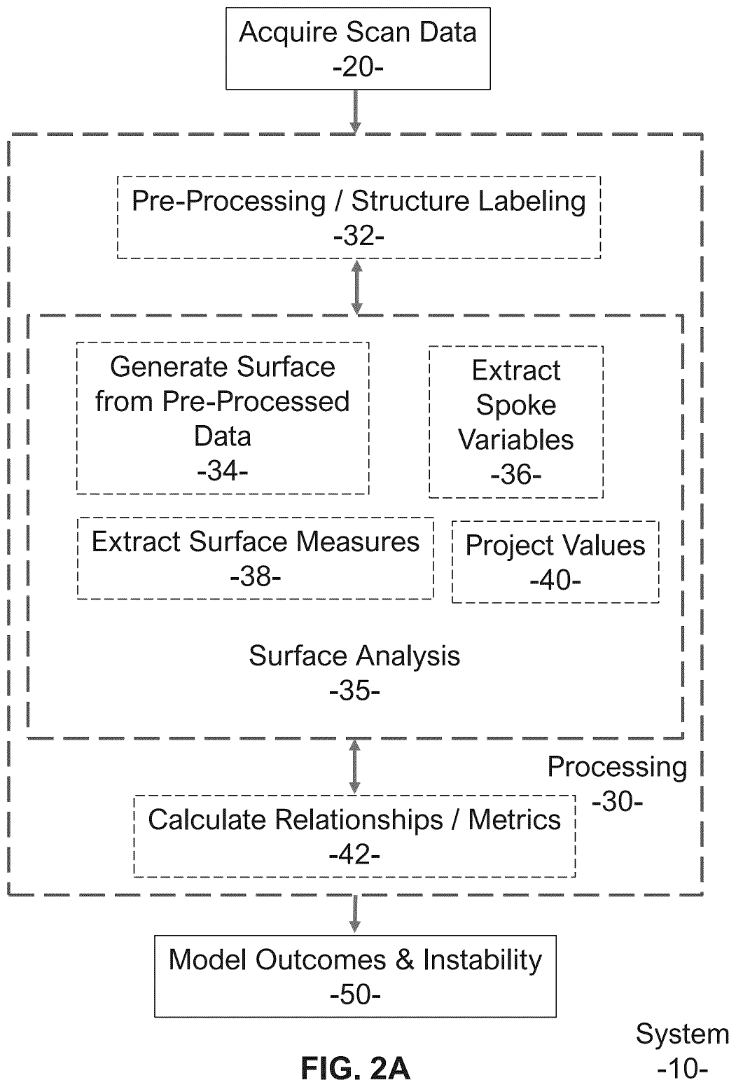

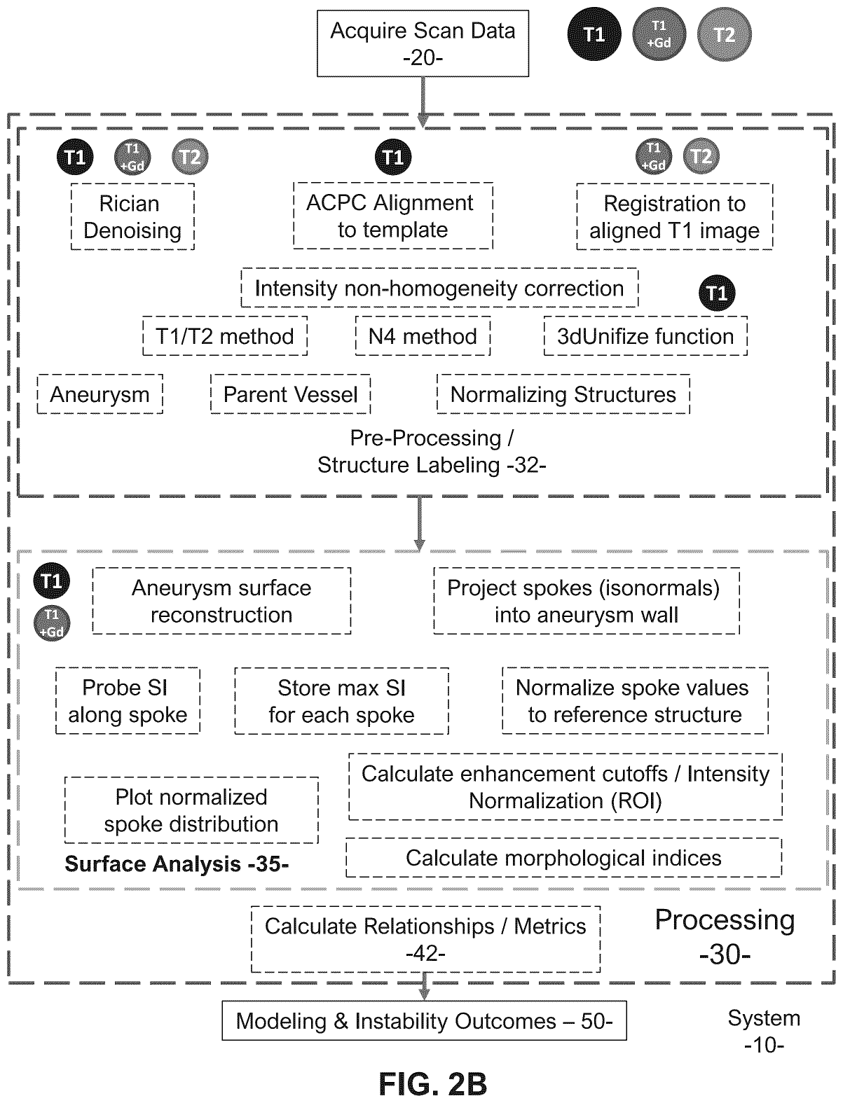

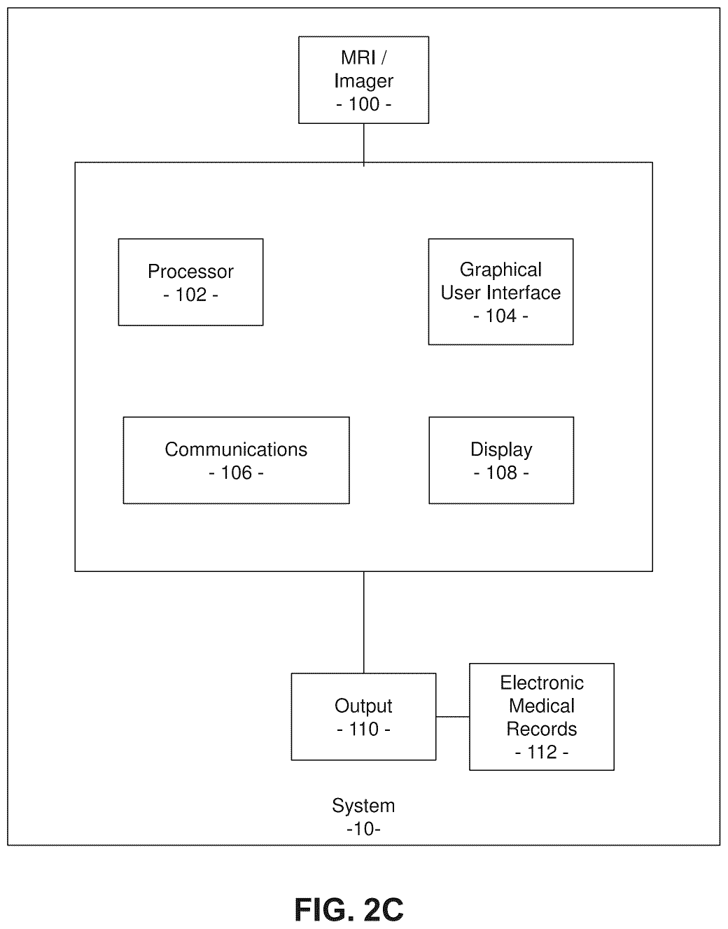

In one exemplary implementation of the disclosed system 10, one step is the acquisition of a scan, as shown in FIG. 2A at box 20, via an imager (FIG. 2C, box 100). That is, in various implementations, scan data such as MRI scan data is inputted into the system 10, such as from a subject. It is appreciated that in various implementations, the scan data comprises certain image data types such as T1, T1+Gd, T2-weighted, Susceptibility-weighted, and Time-of-Flight sequences, for example. Further sequences are of course contemplated and would be understood by those of skill in the art. In various implementations more than one scan is obtained, such as a scan before and after administration of contrast.

In various implementations, another optional step comprises processing scan data (box 30), such a via a processor (FIG. 2C, box 102) as would be appreciated and understood by those of skill in the art. In certain implementations, processing (box 30) comprises one or more optional sub-steps, shown in one illustrative implementation inside box 30. It is appreciated that in certain implementations, various of the steps disclosed in the implementations of FIG. 2A-B are optional and can be omitted, and that in further implementations, additional sub-steps can be included, as would be readily appreciated by those of skill in the art.

In the implementations of FIG. 2A-2C, in one optional processing (box 30) step, the scan data is image cleaned or pre-processed and/or structure labeled (box 32), optionally on a processor (box 102). One illustrative implementation of this pre-processing/structure labeling sub-step (box 32) comprises a variety of optional pre-processing sub-steps, certain non-exhaustive examples including: denoising; alignment of a base image to a template, which can be done rigidly; co-registration to the aligned base image (within-subjects); brain extraction, such as by removing non-brain tissues like the skull, scalp and the like; intensity non-homogeneity correction, such as via N4, or in the case of 7T image data, via 3dUnifize; and region of interest labelling, certain regions of interest being aneurysm, corpus callosum, and the like. Further pre-processing sub-steps are of course possible, and each of the described pre-processing sub-steps is of course optional. In various implementations, a physician, radiologist, or other practitioner can view and/or interact with the scan data via a graphical user interface (GUI) (box 104) and display (box 108).

In various implementations, communication between the various components of the system 10 is effectuated via one or communications devices or components (box 106). Such communication may be wired, wireless, or any other communication technology recognized by those of skill in the art.

Continuing with the implementation of FIG. 2A, in another optional processing (box 30) step, the scan data undergoes surface analysis (box 35), which can comprise a number of optional sub-steps.

In the implementation of FIG. 2A, the pre-processed scan data undergoes aneurysm surface generation (box 34). In various of these implementations, the aneurysm surface generation (box 34) comprises one or more optional sub-steps, such as: loading voxel data; calculating one or more bounding boxes on identified regions of interest; interpolating voxel intensity values, including up-sampling and/or interpolating isotropic voxel size; processing regions of interest, such as via smoothing via 3D Gaussian filter and/or interpolating voxels such as up-sampled and isotropic voxels; converting region of interest voxels to polygon surface; reducing surface complexities, i.e., simplifying the polygons; calculating surface isonormals (called spokes) at each face of surface polygon; limiting the spokes to extend externally; and interpolating intensity along the spoke or spokes. it is appreciated that the spokes according to these implementations can be tailored to the thickness of the wall, which increases the accuracy of tissue sampling. Further sub-steps are of course possible, and each of the described sub-steps is of course optional.

Continuing with the implementation of FIG. 2A, during processing (box 30 according to another optional surface analysis (box 35) sub-step, variables such as spoke variables are extracted (box 36). In these and other implementations, the variable extraction (box 36) comprises a variety of optional sub-steps which can be executed in any order, certain non-limiting examples being: determining intensity distributions along the spoke or spokes; measuring central tendencies and/or other distribution parameters or properties, such as mean, max, median, minimum; identifying position of surface surrounding identified regions of interest, different methods using constant thickness, Laplacian-like localization; and calculating local curvature of surface, such as mean curvature and Gaussian curvature. Further variable extraction step (box 36) sub-steps are of course possible, and each of the described sub-steps is of course optional.

In further optional processing and surface analysis (box 35), the system 10 extracts surface measurements (box 38) such as a region of interest and surrounding 3D surface measures. In these and other implementations, certain implementations utilize one or more sub-steps, certain non-limiting examples including: tetrahedral interpolation of surface triangulation; converting surfaces to convex hulls; extracting variables from all surfaces (region of interest and surround, standard and convex hull), including surface area, volume, ellipticity, non-sphericity and/or undulation. Further surface extraction (box 38) sub-steps are of course possible.

In a further optional aspect of surface analysis (box 35) shown in the implementation of FIG. 2A, the system 10 projects values (box 40) on the region of interest and/or surrounding surface. in various implementations, for example, the system 10 allows for the generation of histogram data for detailed thresholding of wall intensity. In these implementations, the data generated through histogram analysis allows quantification of individual tissue enhancement and the comparison of enhancement with other tissues. It is appreciated that in these implementations, it can also allow for the mapping of focal areas of increased enhancement and the calculation of the relationships between the scan data.

That is, in yet a further optional processing (box 42) step, the system 10 calculates relationships/metrics (box 42) between local measures, metrics, and the other processed data as the understood metrics described further below in greater detail in the provided Examples. Various metrics including diameter, size ratio, aspect ratio, ellipticity index, non-sphericity index, undulation index, surface area, volume, wall thickness, bleb percentage, mural thrombosis, signal intensity among others as would be understood. Further metrics include AWE, GAWE, FAWE, CAWE, and SAWE as determined by comparing calculated metrics to SDs and other threshold values.

As shown in the implementation of FIG. 2A, the system 10 optionally models outcomes (box 50) on the basis of the scanned data and calculated relationships/metrics, which, in certain implementations, can be done based on distributions and the relationships calculated in extracted measures as determined by the defined metrics and thresholds. Again, further discussion of these aspects is discussed below. In various implementations, the outcomes (box 50) may be outputted (FIG. 2C, box 110) for analysis or interpretation by a practitioner such a physician. In some implementations, the outcomes (box 50) may be entered into a patient's electronic medical records (FIG. 2C, box 112). Various outcomes include likelihood of rupture, growth, stroke, or other clinical sign/symptom as would be understood. Additional outcomes may include likelihood of stability, where no treatment may be required.

FIG. 2B shows a further implementation of the various steps and sub-steps according to possible implementations of the system 10. In this implementation, and as described below, several optional steps and sub-steps are possible, and can optionally be tailored to specific scan types, as would be appreciated by those of skill in the art. It is further appreciated that the various described steps are not only optional but may be performed in any order, and can be performed iteratively or reciprocally, as would be understood by those of skill in the art. That is, certain surface analysis (box 35) steps or sub-steps may be performed before certain per-processing/structure labeling (box 32) steps or sub-steps, as required by the executing software.

Briefly, in the exemplary implementation of FIG. 2B, the system acquires scan data (box 20) and executes one or more processing steps (box 30) comprising pre-processing/structure labeling (box 32) and surface analysis (box 35).

According to the implementation of FIG. 2B, during the pre-processing/structure labeling aspect (box 32), the scan data can be processed via a number of optional sub-steps, certain non-limiting examples being: denoising via Rician denoising or other equivalent denoising approach; aligning to a template such as via anterior commissure/posterior commissure (ACPC) alignment; registration aligning to a T1 template where applicable; corrected for intensity non-homogeneity via a number of processes; further, the T1/T2 method, N4 method, or 3dUnifize functions may be performed; the structures normalized; and the structures labeled, such as aneurysm, parent vessel, and the like. Various implementations will of course include alternate steps and the applicable steps, while omitting those that are not applicable, such as on the basis of scan type and the like.

Continuing with the implementation of FIG. 2B, the system 10 further executes one or more optional surface analysis (box 35) steps, certain optional non-limiting examples including: aneurysm surface reconstruction, spoke (isonormal projection into the aneurysm wall; probing the surface intensity (SI) along the spokes; storing the max SI for the given spokes; normalizing the spoke values to a reference structure such as the corpus callosum (CC), as described in detail below; plot normalized spoke distribution; calculating the enhancement cutoffs, that is the intensity normalization for a region of interest such as the CC; and/or calculating the morphological indices, as well as further optional steps/sub-steps that would be readily appreciated by those of skill in the art. Various of these optional steps including the generation of metrics for further analysis or comparison as discussed further herein and in the provided Examples.

According to implementations like that of FIG. 2B, the relationships can be calculated (box 42) for modeling and assessment of the instability outcomes (box 50) as is also described below in the Materials and Methods of Example 1 and elsewhere in the Examples. Those of skill in the art would readily appreciate the various software, hardware, and firmware components necessary to effectuate the described process steps and achieve the desired outcomes, as further described below.

Turning to FIG. 2C, in various implementations an imager, MRI, or other scan generator (box 100) generates one or more scan and data of an artery or other area of interest. In various implementations, multiple scans are generated of the same area including scans with and without interest. In these and other implementations, the data generated from the imager (box 100) is processed via one or more processors (box 102). Various processing steps are discussed herein in relational to FIGS. 2A and 2B.

In various implementations, practitioners can interact with, via, and/or manipulate scan data via a graphical user interface (box 104) and/or a display (box 108).

Continuing with FIG. 2C, scan data, manipulated/processed data, outcomes, and other information may be transferred between system 10 components via one or more communications systems or devices (box 108). Communications system and devices (box 108) include both wired and wireless systems and devices and any communications method recognized by those of skill in the art.

The processed data may optionally output (box 110) certain data including, but not limited to detection of brain aneurysms that are likely to grow and/or rupture, detection of brain aneurysms that are stable and may not require treatment, detection of arterial brain segments that may cause strokes, detection of inflamed or otherwise diseased arteries, and determination of stroke risk as indicated by the metrics and/or statistical analysis as discussed herein. That is, in an optional compare/metrics determinations step the processor or other components uses the processed scan data/normalized morphology chrematistics/other metrics which can then be compared to established standards to assess deviations and the correlated risks of negative clinical outcomes. The outputs (box 110) may be read and analyzed by a practitioner. The outputs (box 110) may be inputted into electronic medical records (box 112) or other record keeping. Optionally, the outputs (box 110) may dictate or facilitate treatment to screen and detected conditions.

### EXPERIMENTAL EXAMPLES

The following examples are put forth so as to provide those of ordinary skill in the art with a complete disclosure and description of how the articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Image Acquisition and Processing. After institutional review board approval patients with unruptured intercranial aneurysms (UIAs) underwent HR-MRI on a GE 7T MRI (Discovery MR950) from August 2018 to December 2020. T1-weighted (sagittal CUBE 3D acquisition, matrix=324×512×512, FOV=256×256 mm, voxel size=0.4×0.4×0.4 mm, slice spacing=0.4 mm, TR=0.7, TE=0.01041 s, flip angle=90°) and T2-weighted images (sagittal CUBE 3D acquisition, matrix=162×256×256, FOV=256×256 mm, voxel size=1×1×1 mm, slice spacing=1 mm, TR=2.5 s, TE=0.071837 s, flip angle=90°) were acquired. T1-weighted images were acquired before and after administering Gd. All image processing was completed with Advanced Normalization Tools and the FMRIB Software Library, as well as custom scripts. DICOM datasets were converted to NIfTI-1 format for pre-processing. All images were denoised using a Rician distribution model. T1 images were aligned to a template brain to approximate anterior commissure/posterior commissure alignment. T2 and T1 post-Gd were then co-registered using rigid, affine, and symmetric normalization to the aligned T1 pre contrast image. Intensity non-homogeneity in T1 was corrected using three sequential procedures: the T1/T2 method, the N4 method, and the 3dUnifize function in the AFNI toolbox as well as custom scripts. It is appreciated that these processing steps (see FIGS. 2A-2B, box 30) and sub-steps are optional and can be omitted or substituted for various implementations, and that the various steps described herein represent one exemplary implementation of those steps and sub-steps.

Structure Labeling and Measurement. Masks of each aneurysm were generated with 3D Slicer on post-processed T1 and T1+Gd images. Binary label maps were then created and resampled to the base image. Additionally, the genu of the corpus callosum (CC) was marked as a 3 mm spherical region of interest. Another region of interest was selected in the pituitary stalk. Stray voxels were isolated and removed from all labels. Average aneurysm wall thickness was measured on source imaging using the Picture Archiving Communication System (Carestream Vue PACS, Rochester, NY). Additionally, mural thrombosis was identified as high T1 signal.

Semi-Automatic SI Quantification and 3D AWE Reconstruction. The signal intensity (SI) of the aneurysm wall was mapped from T1 and T1+Gd images using image processing tools and custom scripts in MATLAB 2020a (The MathWorks, Natick, MA). Base images were isotropically resampled using a cubic interpolation method. A bounding box was created with a 25 mm radius from the aneurysm and vessel labels. Labels were smoothed using a 3 mm gaussian kernel with a 1 mm standard deviation (SD). Larger aneurysms were smoothed with a 5 mm or 7 mm kernel depending on size. Isosurfaces were created from the smoothed and interpolated labels. Surface complexity was reduced to 1 or 10% of the original surface for medium and large aneurysms. Small aneurysms (<5 mm) maintained their original surface complexities.

For each vertex in the aneurysm surface model, isonormals were calculated, creating spokes extending outward from the previously generated masks at a distance equivalent to the average aneurysm wall thickness. Parent artery labels were used to exclude spokes created at the neck of the aneurysm. Along each spoke, the SI from the base image was calculated using a cubic spline interpolation. The maximal value of each spoke was used to determine the SI of the wall and to eliminate luminal contamination. The raw SI values for each spoke were normalized to the max SI values of the CC and pituitary stalk. 3D heat maps were created by superimposing the SI data on the outer aneurysm surface as determined by the outermost vertex of each spoke. AWE was calculated as the mean value of each spoke.

Semi-automatic Bleb Analysis. Blebs were identified based on previously identified criteria. Average bleb wall thickness was calculated as the average of five measurements in the cardinal planes. Blebs were then manually labeled on the processed T1+Gd images according to the same protocol described for aneurysms. Bleb raw values were also normalized to the CC and pituitary stalk.

Manual SI Quantification. Manual SI for regions of interest were sampled to compare the semiautomated 3D mapping method with the manual method of determining AWE. The manual method has been used extensively by various persons in the art to quantify AWE. Wall enhancement of each aneurysm was measured with Picture Archiving Communication System (Carestream Vue PACS, Rochester, NY). AWE was quantified by creating regions of interest of the aneurysm wall in each plane on source T1+Gd sequences as previously described. Regions of interest were sampled in three different planes and after co-registration of the T1+Gd and T1 sequences. Normalization to the pituitary stalk or CC was calculated as follows: (Mean or Max SI_{wall,post})/(Mean or Max SI_{pituitary stalk} or SI_{CC}). Similarly, bleb regions of interest were used to manually determine wall enhancement, and normalized to the pituitary stalk and CC. After normalization with the pituitary stalk, a contrast ratio (CRₛₜₐₗₖ) was obtained.

Morphological Indices. Planar-isolated aneurysm segmentations were created on T1+Gd aneurysm labels using 3D Slicer. These labels were processed with source images using MATLAB to find label volume, surface area, convex hull volume, and convex hull surface area. Ellipticity index, non-sphericity index, and undulation index were subsequently calculated for each aneurysm. Size, size ratio, and aspect ratio were manually calculated from subtraction angiograms, computed angiograms or magnetic resonance angiograms as described by others previously.

Statistical Analysis. All statistical analysis was conducted using SAS (SAS Institute, Cary, North Carolina) and SPSS Statistics 25 (IBM, New York, USA). Categorical variables are presented as frequency and percentage, and continuous variables as mean±SD. A Student t-test was used to compare continuous data, and Pearson chi-squared test for the relationship between categorical data. The manual and semi-automated methods for measuring SI were compared for agreement using Pearson's correlation. Bleb SI was compared to aneurysm SI using the Wilcoxon Signed Rank Test. Logistic regression analysis was used to determine the association of aneurysm instability based on size >7 mm with morphological and AWE predictors. Odds ratios and 95% confidence intervals were constructed for each predictor. A receiver operating characteristic analysis was performed with cutoffs calculated according to the Youden Index. All statistical tests used two-tail alternatives and assessed significance at α=0.05.

### Results

Thirty patients with 33 unruptured intercranial aneurysms were included in the analysis: 28 saccular and 5 fusiform aneurysms. The average aneurysm spoke length based on wall thickness was 0.55±0.09 mm and the average surface area/aneurysm was 311.8±680 mm². An average of 62 spokes per mm² were generated. Only saccular aneurysms were included in the morphological analysis (Table 1) as it has been described that fusiform aneurysms undergo different biological processes. The entire cohort was included in validation of the AWE semi-automated method.

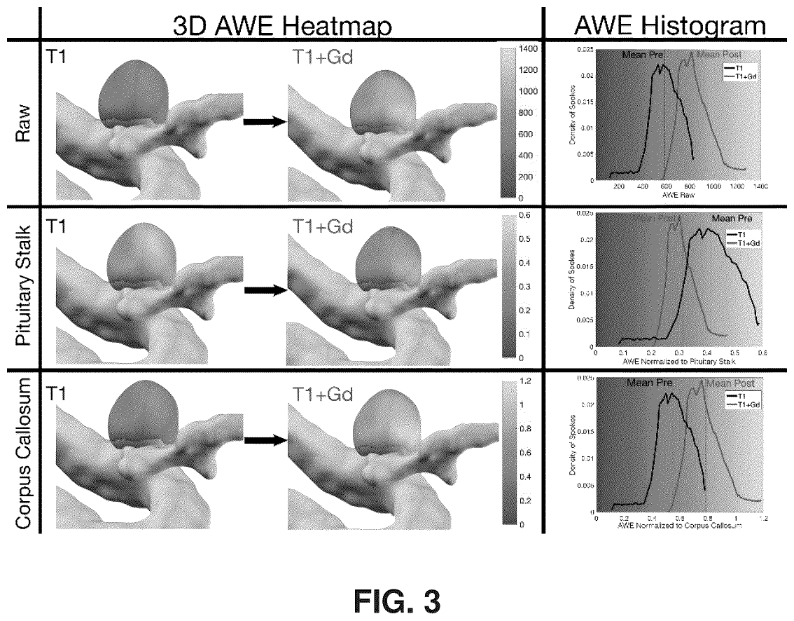

Measurement of AWE and selection of normalizing structure. Comparisons between manual and semi-automated methods showed a correlation for CC and pituitary stalk ratios (r=0.792, r=0.806, p<0.001). Histogram analysis showed that normalization to the CC provided a more reliable shift of the curve from lower AWE in T1 sequences towards higher AWE in T1+Gd sequences. In some cases, avid enhancement of the pituitary stalk introduced artifact and the curve shifted to the left (FIG. 3). As a result of this artifact, 58% of pituitary stalk ratios were negative (T1+Gd<T1) versus 3% of CC ratios. AWE displayed as the shift of CC ratio from T1 to T1+Gd was also significant (p=<0.0001), as opposed to the pituitary stalk ratio (p=0.10) (FIG. 3, Table 2). The coefficient of variation of the CC was lower (28%) than the pituitary stalk (38%) (Table 2). Therefore, despite previous data, the CC appears to be a more reliable normalization structure in determining AWE (Δ T1+Gd−T1) within the same aneurysm.

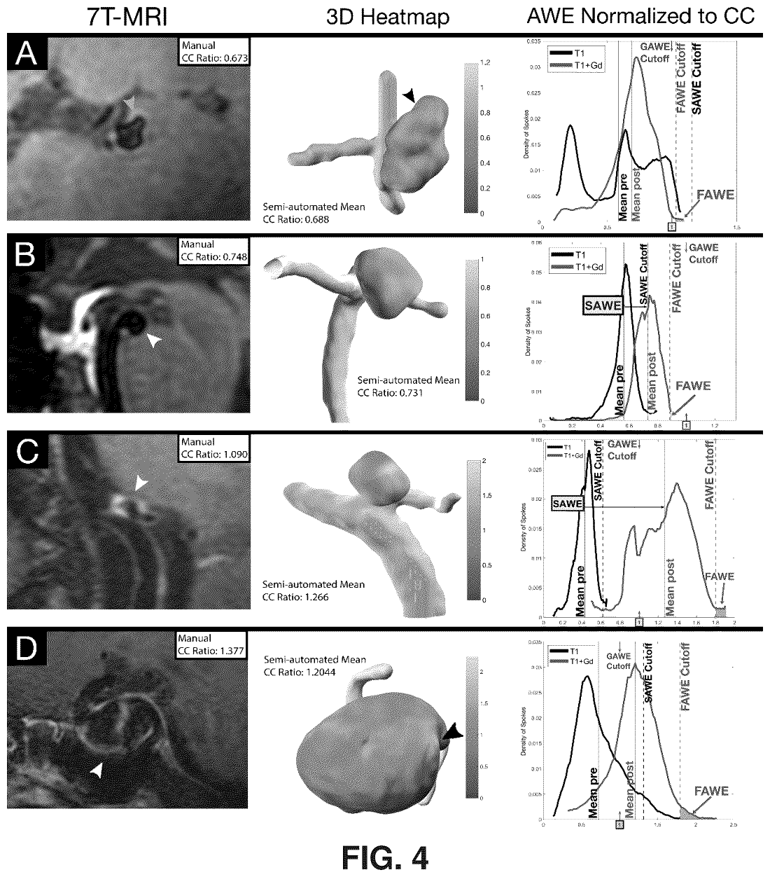

Histogram Analysis and Definitions of AWE. For each aneurysm, the distribution of spokes was plotted according to SI ratios of pre- and post-contrast images. Three metrics of AWE were determined: 1) Specific AWE (SAWE), defined as the change in enhancement between T1 and T1+Gd for each aneurysm (T1+Gd μ≥T1μ+2σ). 2) General enhancement (GAWE), defined as the average CC ratio of all SAWE+ aneurysms (μ=1.0023) or CC Ratio≥1. GAWE was used to compare AWE between different aneurysms. 3) Focal enhancement (FAWE), defined as areas of high AWE with two SDs above the mean of the T1+Gd distribution (≥T1+Gd μ+2σ) (FIG. 4).

SAWE, GAWE, and FAWE analysis. Eleven aneurysms were SAWE+ and had a higher percentage increase in AWE from T1 to T1+Gd (μ=88%), when compared to non-enhancing aneurysms (μ=47%, μ=0.008). Moreover, enhancing aneurysms had a higher increase in CC ratio (μ=0.46) from T1 to T1+Gd vs non-enhancing aneurysms (μ=0.22, p<0.001). The average CC ratio for enhancing SAWE+ aneurysms was 1.002 (p=0.001). Seven aneurysms were GAWE+ and had a higher percentage increase in CC ratio from T1 to T1+Gd (μ=96.3%) vs non-enhancing aneurysms (μ=52%, p=0.012). Enhancing GAWE+ aneurysms also had a higher CC ratio increase (μ=+0.56) between T1 and T1+Gd when compared to non-enhancing aneurysms (μ=+0.24, p<0.001) (Table 1). Twenty-three aneurysms (82%) displayed FAWE. Of these aneurysms, 15 (65%) had only one area of FAWE. Aneurysms that had more than one area of focal enhancement (n=8, 35%) had a larger area under the curve above two SDs (μ=0.035, p=0.005). Histograms of aneurysms with more than one area of FAWE had a positive skew (μ=0.315, p=0.04). Histograms of aneurysms that had only one area of FAWE had a negative skew (μ=−0.430, p=0.04).

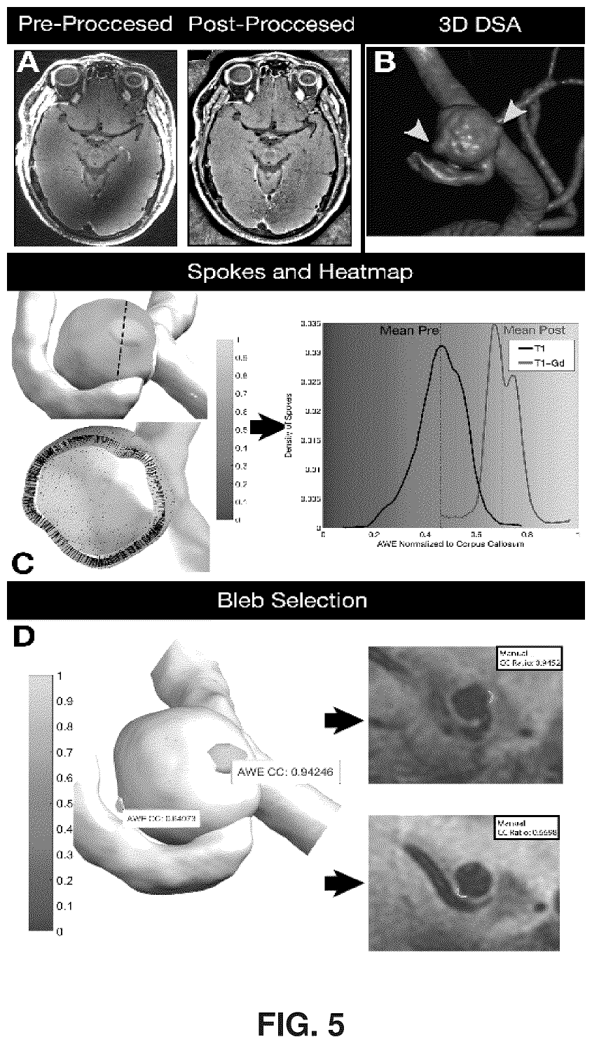

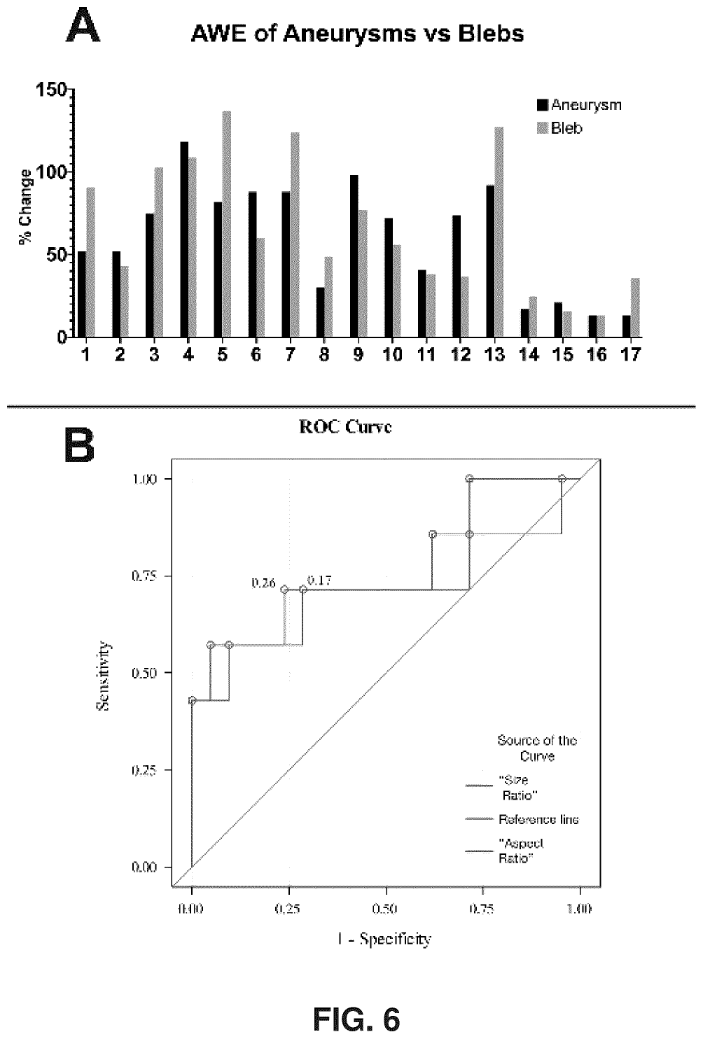

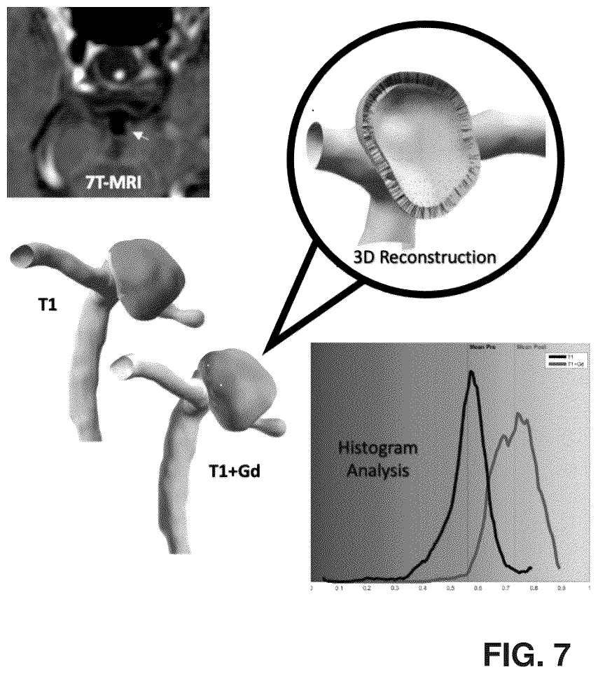

3D Morphological Analysis. 3D-AWE maps showed the same morphology as depicted in 3D rotational angiograms (FIG. 5). This allowed a separate and detailed analysis of the aneurysmal sac versus blebs. The maximal AWE was significantly different between the sac and blebs (P=0.0017). Nine of 17 aneurysm (53%) with blebs exhibited increased AWE in the bleb, compared to the sac, mean AWE increased 8% (FIG. 6A). Two-tailed Student's t-tests of morphological characteristics demonstrated that GAWE+ aneurysms had a thicker wall (0.62±0.1 mm) than non-enhancing aneurysms (0.51±0.09 mm, p<0.008). Size (12.39±8.13 mm, p=0.049), size ratio (3.95±2.59, p=0.01) and aspect ratio (3.63±2.35, p=0.002) were also significantly different between GAWE+/− aneurysms (Table 1).

Bivariable logistic regression analysis for aneurysms ≥7 mm showed as predictors of instability: size ratio≥2.16 (Odds Ratio 23.6, 95% Confidence Interval 2-278) and aspect ratio≥2.31 (Odds Ratio 7.88, 95% Confidence Interval 1-45). These morphological parameters have also been identified by other groups as a reliable indicators of instability. In establishing AWE as a CC ratio with a threshold ≥1 (GAWE+), bivariate analysis showed significance for aspect ratio (Odds Ratio 2.23, 95% Confidence Interval 1-4) and size ratio (Odds Ratio 1.90, 95% Confidence Interval 1-3). In receiver operating characteristics analysis, aspect ratio≥3.56 (specificity 95%, sensitivity 57%) and size ratio≥2.89 (specificity 76%, sensitivity 71%) best predicted GAWE+ aneurysms. (FIG. 6B).

### Discussion

This study performed a detailed topographic analysis of AWE images acquired with 7T HR-MRI. A protocol was developed to generate and analyze 3D-AWE maps of the entire aneurysm. With this tool an entire set of new metrics could be objectively analyzed and used to better understand the distribution of AWE along the aneurysm wall. This approach will allow detailed analysis of wall tissue from MRI images.

The generation of 3D color maps of AWE may become a powerful tool in quantifying enhancement and better understanding aneurysm biology. Previously 25 aneurysms were analyzed and the presence of partial or complete enhancement was subjectively determined coupled with hemodynamic simulation and morphological measurement for instability analysis. Isonormals were used to measure AWE normalized to the nominal intensity of the image volume.

This prior approach fell short of adding objective parameters of determining AWE. The currently disclosed method and system generated thousands of datapoints (μ=4,490 spokes/aneurysm) from orthogonal spokes projected into the aneurysm wall axis. The measurement of AWE was optimized with 7T HR-MRI by tailoring the spoke length to the thickness of the aneurysm wall for each aneurysm (range 0.38-0.78 mm). Customized sampling of AWE decreases artifact introduced by low and high enhancing structures such as the cerebrospinal fluid and the cavernous sinus, respectively.

Others have demonstrated on 7T MRI that the radiological aneurysm wall thickness can vary between 0.2-1.6 mm. A tailored spoke length generates exceptionally reliable 3D maps of AWE. This approach has several advantages over previously described methods of AWE analysis: 3D data allows identification of areas of aneurysm instability such as blebs, the aneurysm is visualized and analyzed in its whole magnitude, and histograms generated through this protocol provide a new tool in studying the biology of brain aneurysms and in identifying possible markers of instability. Meta-analysis and preliminary prospective data have shown that AWE is a potential predictor of aneurysms instability.

However, there is no consensus on how to determine thresholds of AWE or what part of the aneurysm should be sampled to analyze enhancement. 3D maps may also provide further information about the aneurysm biology as it has been shown that aneurysm compartments have different patterns of enhancement directly influenced by local flow conditions. 7T MRI studies have demonstrated a linear correlation between wall thickness and SI. Histological analysis of aneurysm walls has shown an eight-fold variation in thickness. Different biological processes encompass this wide variation in thickness and structure of the aneurysm wall. Atherosclerotic and non-atherosclerotic calcification, inflammation, wall remodeling with mural necrosis, proliferation of vasa-vasorum, among other changes, will determine the “health” of the aneurysm wall and risk of rupture. A 3D AWE map analysis may provide a comprehensive insight into the heterogenous processes that lead to aneurysm growth and rupture.

Assessment of AWE has been performed subjectively by most groups, which limits reproducibility and affects accuracy. Even the so call “objective methods” of AWE analysis introduce subjectivity since regions of interest are manually drawn to select areas of the aneurysm deemed as “enhancing”. To standardize the analysis of AWE, multiple enhancement metrics were previously analyzed and it was determined that a pituitary stalk ratio≥0.60 had a sensitivity of 81% in detecting aneurysms ≥7 mm in diameter. This threshold correlated well with clinical predictive scales of aneurysm growth and rupture.

AWE analysis was previously performed in multiplanar 2D reconstructions at best and did not capture the 3D structure of the aneurysm. This objective metric for AWE, provided a framework for the described objective 3D analysis. A strong correlation was determined between the 3D method and manual multiplanar analysis (r=0.792). Previous studies suggested that the pituitary stalk is a reliable normalizing structure. However, in this study the pituitary stalk exhibited an avid uptake of Gd in T1+Gd images, which is suboptimal when analyzing specific AWE (SAWE). Other studies have successfully used white matter structures for normalization of enhancement through pre/post Gd indexes. After multiple comparisons and detailed histogram analysis, normalization to the CC resulted in a more reliable parameter in determining AWE. (FIG. 3).

The generation of AWE histograms provided a new set of metrics for analysis. Specific thresholds were determined based on histogram analysis: General AWE (GAWE), Specific AWE (SAWE), and Focal AWE (FAWE). Coupling this data to 3D reconstructions allowed qualitative and quantitative assessments of AWE. (FIG. 5). Interestingly, aneurysms with morphological parameters suggestive of stability, but with documented growth, were both GAWE and SAWE positive (FIG. 4, Panel C). The threshold for SAWE was determined as a shift of CC ratio of at least two SDs from the mean enhancement on T1 images. This approach allowed the analysis of Gd uptake by each aneurysm. An aneurysm-based analysis would be required in determining risk of rupture in patients with multiple aneurysms. GAWE was defined as a CC ratio≥1 on T1+Gd images. This threshold was used to compare different aneurysms. Some aneurysms that were GAWE+, did not meet the threshold for SAWE (FIG. 4, Panel A). Other aneurysms that did not appear enhancing when compared to other aneurysms (CC ratio<1), were SAWE and FAWE positive. (FIG. 4, Panel B). Others have previously used pharmacokinetic modeling to estimate the contrast extravasation rate (Kₜᵣₐₙₛ) in identifying aneurysms that ruptured over time. This appeared to be a more reliable metric than the analysis of enhancement determined by T1+Gd images only. SAWE may be a better tool in detecting “leaky” aneurysms when compared to GAWE. SAWE provides a map of enhancement for each aneurysm, which is an individualized metric inherent to Gd uptake in each segment of the wall. Moreover, SAWE has the potential of controlling for enhancement artifacts such as slow-flow, as histogram analysis of T1 and T1+Gd images has the potential of identifying pseudoenhancement (FIG. 4, Panel A).

The generation of AWE heat maps allowed the identification of areas of increased enhancement including blebs. Others have previously shown that areas of focal enhancement have low wall-shear stress conditions that may favor growth and rupture. 3D-AWE maps ease identification of these areas of focal enhancement which on conventional 2D imaging may not be detected. An objective threshold for FAWE was determined by detailed histogram and 3D map analysis. The highest SI region in the histogram could identify both the threshold for FAWE and whether there are multiple areas of FAWE in the aneurysm sac (μ=0.035, p=0.005). Aneurysms with larger areas of FAWE also had histograms with positive skew, suggesting that these highly enhancing focal areas can affect the shape of the histogram. FAWE has been linked to intraluminal thrombus and sites of rupture, this analysis could further identify and quantify these areas. 3D AWE maps showed that on average, blebs exhibited 8% higher enhancement than the aneurysm sac. Further topographic analysis may lead to the identification of bleb-prone areas in the aneurysm sac.

3D AWE mapping may be a powerful tool in studying the biology of brain aneurysms and in identifying aneurysms that may grow and rupture. This method generates a new set of metrics which could potentially be correlated with different biological processes of the aneurysm wall.

### Example 2

### Methods

Semi-Automatic SI Quantification and 3D AWE Reconstruction: After institutional review board approval, patients with unruptured intracranial aneurysms underwent HR-MRI on a GE 7T MRI (Discovery MR950) from August 2018 to December 2020. (Table 3). Using a method for 3D AWE quantification, manual segmentations of the aneurysm sac were created from registered T1 and T1+Gd images. Spokes were extended outwards orthogonal to the surface to probe the aneurysm wall. The spoke length was determined by a distance equivalent to the average radiological wall thickness measured on T1+Gd imaging. The signal intensity (SI) of each spoke was used for analysis after normalization with the genu of the corpus callosum (CC):

CCᵣₐₜᵢₒ=SI_{max spoke}/SI_{mean CC}

These values were subsequently mapped to the aneurysm surface to create 3D AWE heatmaps.

Histograms of the AWE data were created over 100 bins for T1 and T1+Gd reconstructions. Histogram shape was characterized using skewness, kurtosis, and standard deviation (σ).

Classifications of AWE: The circumferential SI (μ) was calculated by averaging the values from each spoke over the entire aneurysm surface.

Circumferential SI:ρ=ΣCCᵣₐₜᵢₒ/Nₛₚₒₖₑₛ

Specific aneurysm wall enhancement (SAWE) was used to describe the dynamic uptake of contrast between T1 and T1+Gd imaging. SAWE+ aneurysms were defined as having greater than two standard deviations (SDs) of circumferential SI on T1+Gd when compared to T1 (μₚₒₛₜ≥μₚᵣₑ+2σ).

$$\{ \begin{matrix}SAWE + : {μ}_{post} \geq {μ}_{pre} + 2 {σ}_{pre} \\ SAWE - : {μ}_{post} < {μ}_{pre} + 2 {σ}_{pre}\end{matrix}$$

General aneurysm wall enhancement (GAWE) was used to characterize enhancement relative to the entire cohort based only on T1+Gd imaging. Aneurysms with an enhancement ratio higher than the CC (μₚₒₛₜ≥1) were classified as GAWE+. Previous studies have established different thresholds after normalization. In this analysis a μₚₒₛₜ≥1 was used as the threshold for enhancement.

$$\{ \begin{matrix}GAWE + : {μ}_{post} \geq 1 \\ GAWE - : {μ}_{post} < 1\end{matrix}$$

Focal aneurysm wall enhancement (FAWE) was used as a measure of regional/focal enhancement when compared to the rest of the aneurysm. Spokes with a CCᵣₐₜᵢₒ greater than 2 SDs above the circumferential SI of the T1+Gd reconstruction were considered FAWE+(CCᵣₐₜᵢₒ≥μₚₒₛₜ+2σ). Areas of FAWE were determined by the density of spokes above this threshold.

$$\{ \begin{matrix}FAWE + : CC Ratio \geq {μ}_{post} + 2 {σ}_{post} \\ FAWE - : CC Ratio < {μ}_{post} + 2 {σ}_{post}\end{matrix}$$

Measurements of Aneurysm Morphology: Fusiform aneurysms were defined as an arterial dilation greater than 1.5 times normal without a clearly definable neck. Size ratio and aspect ratio were measured as defined by Dhar, et al. (Dhar S, Tremmel M, Mocco J, Kim M, Yamamoto J, Siddiqui A H, et al. Morphology parameters for intracranial aneurysm rupture risk assessment. Neurosurgery. 2008; 63:185-196; discussion 196-187.) which is incorporated by reference herein. Mural/intrasaccular thrombosis was identified as high T1 signal, or isointense signal to the normal vessel wall.

Statistical Analysis: All statistical analyses were conducted using SPSS Statistics 27 (IBM, New York, USA). Categorical variables are presented as frequency and percentage, and continuous variables as mean±SD. All statistical tests used two-tail alternatives and assessed significance at α=0.05. Paired categorical data was compared using McNemar's Test. The Shapiro-Wilk test for normality was used to identify normally distributed continuous variables. Normally distributed continuous data were compared using an independent Student's t-test, and non-normally distributed data were compared using a Mann-Whitney U-Test. Comparisons between multiple groups were conducted using a one-way ANOVA with a Tukey post hoc test. Pearson correlations were used to evaluate the linear relationship between normally distributed variables and Spearman correlations were used to evaluate relationships involving non-normally distributed variables.

### Results

Enhancement Metrics: A total of 32 aneurysms were included in the analysis: 24 saccular and 8 fusiform. Six (18.75%) aneurysms had mural/saccular thrombosis. Demographic and aneurysm location data are available in Table 4. Ten (31.25%) aneurysms were classified as GAWE+ and 14 (43.75%) as SAWE+. Eight (25%) aneurysms met both criteria for enhancement (GAWE+/SAWE+), while sixteen (50%) aneurysms did not meet any criteria for enhancement (GAWE−/SAWE−). GAWE+ and SAWE+ saccular aneurysms were larger in size and had higher size and aspect ratios compared to GAWE−/SAWE− aneurysms. (Table 5).

Histogram Shape Metrics in Large Aneurysms: Larger aneurysms (>7 mm, N=14) had significantly higher circumferential SI compared to smaller aneurysms (N=28) on both T1 and T1+Gd reconstructions (p<0.001 and p=0.034 respectively). Larger aneurysms also had significantly higher SDs (histogram width) for T1 and T1+Gd reconstructions (p<0.001 and p=0.022 respectively). Larger aneurysms had less negative (left) skew compared to smaller aneurysms on T1 (p=0.002) and T1+Gd distributions (p=0.068). Positively skewed histograms had larger areas of FAWE and wider distributions. Skewness was correlated with area of FAWE (Spearman r=0.871, p<0.001) and histogram SD (Pearson r=0.510, p=0.003).

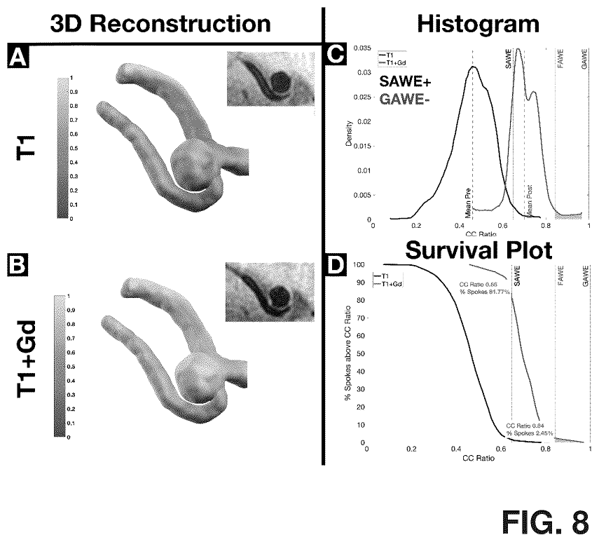

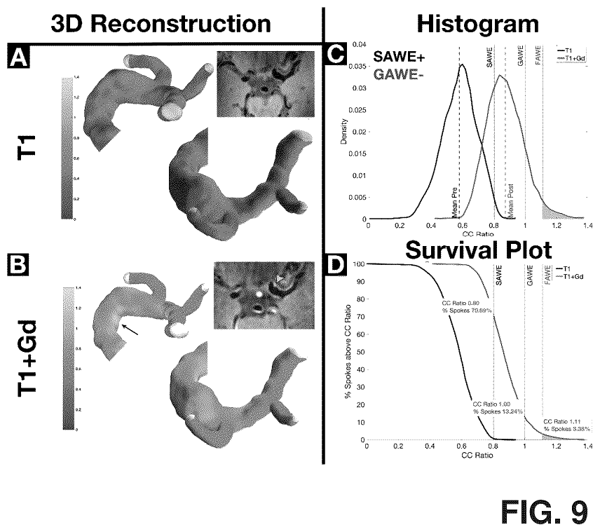

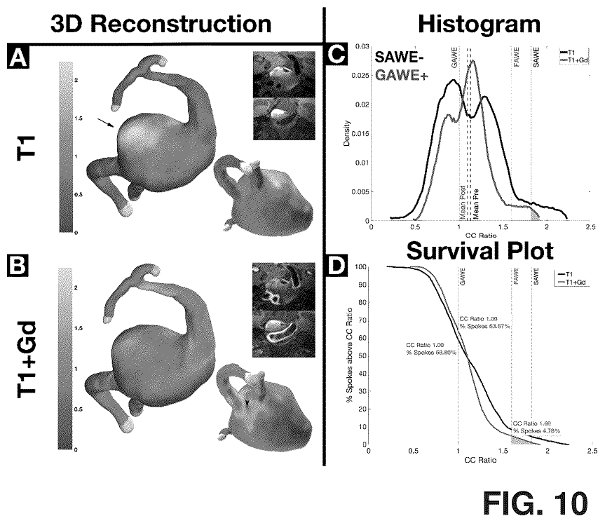

Patterns of Enhancement in Fusiform and Thrombosed Aneurysms: Saccular and fusiform aneurysms displayed different patterns of enhancement. (Table 6). Four (50%) out of the eight fusiform aneurysms were GAWE+ and three (37.5%) were SAWE+. Two (25%) aneurysms were GAWE+/SAWE+, and three (37.5%) were GAWE−/SAWE−. The threshold to define SAWE was higher (p=0.006) in fusiform (μ=1.09) than saccular aneurysms (μ=0.82). All fusiform and 19 saccular aneurysms (79.2%) displayed areas of focal enhancement. Fusiform aneurysms were larger in size, had thicker walls and had more heterogeneous surface enhancement when compared to saccular aneurysms (FIGS. 8B and 9B). Two fusiform (25%) and 4 saccular aneurysms (16.7%) had mural/saccular thrombosis. Some aneurysms with extensive thrombosis had more avid luminal wall enhancement on T1 than T1+Gd imaging and were SAWE− (FIG. 10).

### Discussion

The quantitative data obtained from 3D AWE mapping provides a comprehensive analysis of aneurysm enhancement. Histogram shape metrics, such as skewness and SD provide information about regional enhancement within each aneurysm. 3D AWE mapping encompasses the entire wall in all dimensions and allows identification of subtle enhancement differences that may reflect different biological processes of the aneurysm wall.

Histogram Shape Metrics in Large Aneurysms: The histogram width represents the distribution of enhancement along the entire aneurysm. A larger aneurysm may have more dispersion of enhancement as the wall may undergo different biological process in different compartment. The skewness of the distribution can be used to represent the increased concentration of wall enhancement towards low or high SI. Large areas of focal enhancement located in the right tail of the histogram create a right and positive skew. Aneurysms that exhibited common features of instability (size >7 mm, aspect ratio>1.3 and size ratio>3) had different histogram shapes compared to stable aneurysms. Larger aneurysms (>7 mm) had broader and right-positive skewed distributions, compared to smaller aneurysms. The identification of this pattern of enhancement in smaller aneurysms in a larger sample size may allow the identification of small unstable aneurysms. This is a common paradigm in anterior communicating aneurysms which tend to be smaller at the time of rupture.

Histogram Shape Metrics in Fusiform Aneurysms: The quantification of different enhancement patterns through the analysis of histogram shape metrics established clear differences between saccular and fusiform aneurysms (Table 6). Fusiform aneurysms had higher circumferential SI on T1+Gd imaging and larger areas of focal enhancement, leading to wider, and positively skewed histogram distributions compared to saccular aneurysms (FIG. 9). 3D AWE maps demonstrated a more heterogenous distribution of enhancement in fusiform aneurysms compared to saccular aneurysm. Fusiform aneurysms also exhibited regional enhancement along the aneurysm body. Thicker walls, larger size and more areas of focal enhancement (3 versus 1 in saccular aneurysms) highly suggest that fusiform aneurysms form and grow due to unique biological processes, as opposed to saccular aneurysms.

General Aneurysm Wall Enhancement (GAWE): A normalized metric for AWE is necessary to compare enhancement between different aneurysms. An AWE cutoff normalized to the pituitary stalk (CRstalk=0.60) in identifying aneurysms >7 mm was previously established. However, this approach was also bounded to manual regions of interest determined on 2D imaging. The pituitary stalk avidly enhances on T1+Gd imaging and introduces artifact into the quantification of AWE. Therefore, in current implementations the CC is used for normalization. The quantification of the degree of enhancement relative to the CC on T1+Gd imaging, is equivalent to the AWE cutoff of 0.60 determined in previous studies. In the current analysis, any aneurysm that enhanced more than the CC (μₚₒₛₜ≥1) was classified as GAWE+. Ten out of 32 aneurysms in our cohort met this criterion. These aneurysms were larger in size and had higher aspect and size ratios. (Table 5).

Specific Aneurysm Wall Enhancement (SAWE): Comparing changes in circumferential SI before and after contrast administration allows for the quantification of aneurysmal Gd uptake. SAWE is an aneurysm specific metric used to quantify contrast uptake by comparing T1 and T1+Gd reconstructions. Omodaka, et. al. (Omodaka S, Endo H, Niizuma K, Fujimura M, Inoue T, Sato K, et al. Quantitative assessment of circumferential enhancement along the wall of cerebral aneurysms using mr imaging. AJNR Am J Neuroradiol. 2016; 37:1262-1266), incorporated herein by reference, defined wall enhancement index as the change in enhancement between T1 and T1+Gd images. This index predicted aneurysm rupture with a sensitivity of 100% and specificity of 69%. In the described implementations, for an aneurysm to be classified as SAWE+, there had to be a substantial increase (2SDs) in circumferential SI after contrast administration (μₚₒₛₜ≥μₚᵣₑ+2σₚᵣₑ).

In this Example, some saccular aneurysms with uniform AWE distribution were classified as SAWE+, even though they appeared subjectively non-enhancing on T1+Gd imaging (FIG. 8). Fusiform aneurysms with heterogeneous AWE distribution could also be classified as SAWE+ after substantial contrast uptake (FIG. 9). The method described herein allows for gauging in detail the amount of enhancement exhibited by each aneurysm and by a particular segment of the aneurysm. In a saccular aneurysm, about 82% of spokes enhanced above the SAWE threshold, while only 2.5% showed focal enhancement. (FIG. 8D). Conversely, a fusiform aneurysm showed a similar broad uptake of contrast with 71% of spokes enhancing above the SAWE cutoff in addition to a larger (4%) area of focal enhancement. (FIG. 9D). SAWE has the potential of translating different phenomena among different types of aneurysm. Saccular aneurysms are more likely to show uniform patterns of enhancement, whereas fusiform aneurysms are more likely to show focal heterogenous patterns of enhancement. Aneurysms with significant heterogeneity on pre-contrast imaging, suggestive of complex vascular processes such as the presence of mural thrombosis, may not be classified as SAWE+ due to inherent enhancement on T1 imaging. (FIG. 10)

Focal Aneurysm Wall Enhancement (FAWE): Areas of focal enhancement (FAWE) have been correlated with areas of instability, bleb formation and higher risk of rupture. Herein, FAWE was visualized on 3D AWE maps and the density of FAWE determined on T1+Gd reconstructions. By comparing the CCᵣₐₜᵢₒ of individual spokes relative to the aneurysm body, the area of focal enhancement could be objectively identified. Spokes that substantially enhanced (>2SDs) compared to the circumferential SI on T1+Gd were considered FAWE+(CCᵣₐₜᵢₒ≥μpost+2σₚₒₛₜ). By identifying the density of spokes in the heatmap that met this criterion, it was identified if there were multiple areas of focal enhancement in the same aneurysm (FIG. 9D). FAWE may identify areas that may be susceptible to rupture. FAWE can be identified both qualitatively in 3D heatmaps and quantitatively using T1+Gd distributions. FAWE also represents the heterogeneous patterns of enhancement that may be present in different biological processes such as atherosclerosis, proliferation of vasa vasorum and/or slow flow conditions. Fusiform aneurysms had more areas of FAWE than saccular aneurysms.

Patterns of AWE in Thrombosed Aneurysms: Thrombosed aneurysms exhibited specific patterns of enhancement that suggest different patterns of Gd penetration in the aneurysm structures. AWE was very heterogenous in the outer and thrombosed wall, while the inner wall closest to the patent lumen exhibited uniform enhancement (FIGS. 10A-B). Contrast dynamics in these complex structures translated into changes in areas of FAWE between T1 and T1+Gd imaging (FIG. 10C). The intramural thrombus may limit the uptake of Gd by the outer thrombosed wall and prevents the detection of SAWE. Histological aneurysm analysis has previously been paired with focal enhancement and it has been suggested that the organization of fresh intraluminal thrombus into a loose matrix allowed for thrombus to retain contrast media. This may explain why these aneurysms are SAWE− and GAWE+. The double-rim wall enhancement visible on 7T-MRI of thrombosed aneurysms has been previously characterized as neovascularization of the inner wall layer adjacent to thrombus and vasa vasorum in the outer wall layer. This pattern is consistent with results herein showing that focal enhancement may be associated with areas of thrombosis that create complex patterns of enhancement on both T1 and T1+Gd imaging (FIGS. 10A-B). Quantitative metrics derived from 3D AWE maps and histogram analysis of the outer wall allow identification of these aneurysms. Increases in skewness or changes in histogram SD after contrast administration may reflect the complex pattern of contrast retention in these thrombosed regions.

### Example 3

Image Acquisition and Processing: Aneurysms were prospectively imaged with HR-MRI between May 2018 and November 2021 after approval from the University of Iowa Institutional Review Board. Saccular aneurysms larger than 2 mm in size were included. Fusiform, thrombosed, or small (<2 mm) aneurysms were excluded. MRIs were performed on a 3T MRI (MAGNETOM Skyra, Siemens); T1-weighted (T1), T1+gadolinium (T1+Gd), quantitative susceptibility mapping (QSM), and time of flight (TOF) sequences were obtained. Specific imaging parameters are described in Table 7. T1 and T1+Gd images were isotropically resampled, denoised, and registered. The range of raw signal intensity (SI) values did not change after processing.

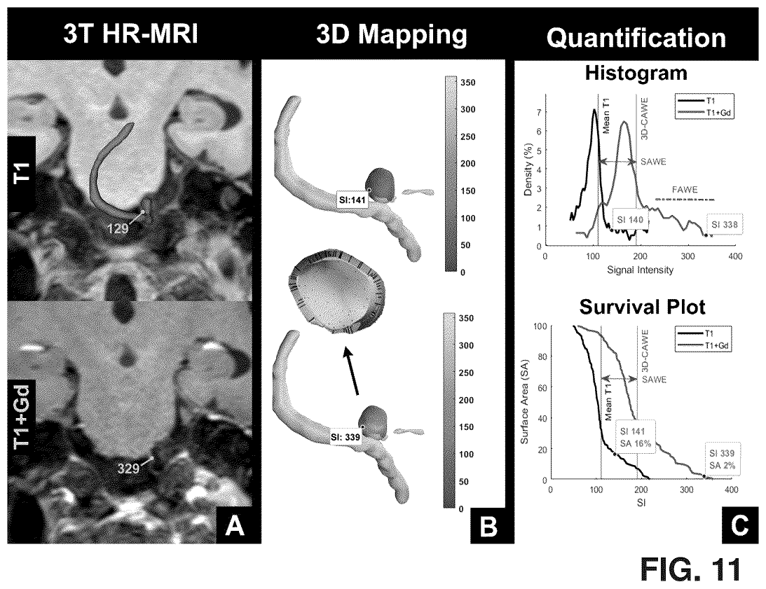

3D Reconstruction and Surface Mapping: A post-acquisition pipeline developed for 7T-MRI was adapted for 3T-MRI. The goal of this method was to generate 3D-AWE maps of the entire aneurysm. Manual segmentations of the aneurysm sac and parent vessel were created with 3D Slicer17 using T1 images. Using custom MATLAB R2021b (MathWorks, Natick, MA) scripts, SI probes were orthogonally extended from the aneurysm lumen 0.5 mm into the aneurysm wall. The number of probes created was proportional to the surface area of the aneurysm. The maximal SI value and spatial location of each probe were used to create 3D surface maps, histograms, and survival plots representing the distribution of SI along the entire aneurysm wall for T1 and T1+Gd images. (FIG. 11). SI values were normalized to the genu of the corpus callosum (CC).

A separate analysis of enhancement in aneurysm blebs was conducted. Aneurysm blebs, identified as thin outpouchings of the aneurysm wall, were isolated from the aneurysm body, and their SI was compared to the rest of the aneurysm body.

Quality Control: To determine the correlation between the herein described 3D-AWE method and prior known methods of measuring AWE, measurements of 3D-AWE to manual 2D measurements were compared in 69 aneurysms. 3D-CAWE normalized to the pituitary stalk (CRₛₜₐₗₖ) was compared to CRₛₜₐₗₖ obtained by manual measurements.

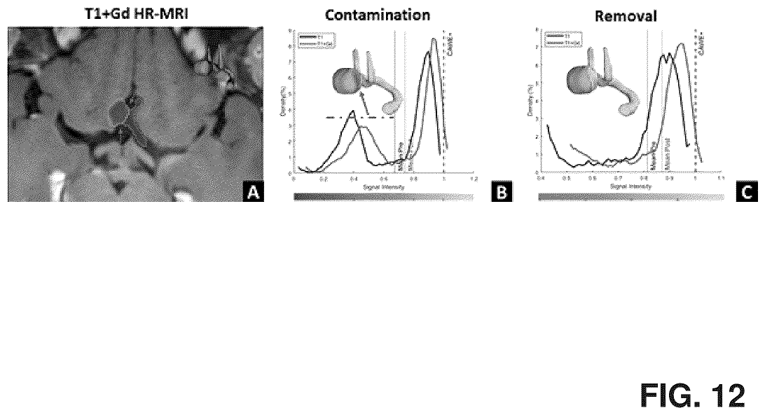

Sampling of structures beyond the aneurysm wall, such as cerebrospinal fluid, bone, or surrounding brain tissue (FIG. 12) was identified by histogram analysis and verified on 3D heatmaps. This artifact was removed by excluding probes with values lower than the 70th percentile of the SI label. Final 3D-AWE reconstructions were visually examined for accuracy, after comparison with source images.

Definitions of Aneurysm Wall Enhancement Metrics: Three metrics were used to describe circumferential, dynamic, and focal patterns of AWE. In 2D, CAWE is used to quantify AWE along the circumference of the wall within one plane. 3D-CAWE samples the entire aneurysm, and is defined by the mean SI of all probes in T1+Gd images. Because SI values were originally normalized to the CC, aneurysms that enhanced (on average) more than the CC (3D-CAWE≥1) were defined as 3D-CAWE+. Aneurysms less enhancing than the CC were therefore classified as 3D-CAWE−. The dynamic uptake of contrast by the aneurysm wall or the change in SI between T1 and T1+Gd has been described in 2D as the wall enhancement index. The 3D equivalent is defined as specific AWE (SAWE), or the difference in mean SI between T1 and T1+Gd. Finally, the presence of FAWE is defined by areas of the aneurysm with increased AWE on 3D-AWE maps. Increased FAWE leads to a positive (right) skewness on histogram analysis.

Classification of Symptomatic Aneurysms: Aneurysms were classified as symptomatic or asymptomatic. Symptomatic aneurysms included ruptured aneurysms, aneurysms accompanied with cranial nerve neuropathy due to pressure or inflammation, and aneurysms that presented with a sentinel headache (unusually severe and sudden within two weeks of presentation).

Morphological Characterization of Aneurysms: Multiple comparisons were performed to determine the relationship between 3D-AWE and morphological metrics. Measurements of largest aneurysm diameter, neck width, sac height, and parent vessel diameter were conducted on either digital subtraction angiography, computed tomography angiography, or magnetic resonance angiography on the best available projection. Aneurysm size (the largest dimension), size ratio (size ratio=ratio of aneurysm sac height to average parent vessel diameter), and aspect ratio (aspect ratio=ratio of perpendicular sac height to neck width) were calculated for each aneurysm. Irregular aneurysms were identified. To evaluate growth or rupture risk using clinical predictive scales, ELAPSS and PHASES scores were calculated for each aneurysm. For dichotomous comparisons of enhancement, aneurysms were categorized as irregular or regular, size ≥7 or <7 mm, high risk location (anterior communicating artery complex, posterior communicating artery, and basilar artery) versus lower risk locations, ELAPSS ≥15 or <15 PHASES ≥5 or <5 and symptomatic versus asymptomatic.

Microhemorrhage Analysis and Reconstruction: Aneurysms found to have microhemmhorage based on QSM imaging were classified as QSM+. Using the MATLAB toolbox, STI Suite, Laplacian-based phase processing was performed on QSM images to produce tissue phase visualization. The subsequent images were then co-registered on 3D-Slicer with TOF-MRA sequences. 3D volumetric reconstructions were conducted by incorporating the microhemorrhage segmentation with the aneurysm at an optimal susceptibility threshold of 0.1 ppm.

Statistical Analysis: Statistical analysis was conducted using either IBM SPSS Statistics for Windows, Version 27.0 (IBM Corp, Armonk, NY) or R, Version 4.1.2 (R Core Team, Vienna, Austria). Categorical data are represented as frequency (percentage). Shapiro-Wilk tests were conducted on continuous data to determine whether they were normally distributed. Normally distributed variables are represented as mean±SD. Non-normally distributed variables are represented as median (IQR). Comparisons between normally distributed data were conducted using Student's t-tests and Pearson correlations. Comparisons involving non-normally distributed data were conducted using Mann-Whitney U tests and Spearman's correlations. Comparisons between unpaired categorical data were conducted using Pearson's chi-squared test, or Fisher's exact test.

To assess the effect of various predictors on the likelihood that a given aneurysm would be symptomatic, a series of univariate logistic regression models were constructed, providing odds ratios, confidence intervals, and p values. Using all variables analyzed in the univariate setting, a multivariable model was constructed using a forward stepwise selection approach oriented toward minimizing the Akaike Information Criterion of the model. At each step of this selection procedure, candidate variables were assessed to see which one would most greatly reduce the Akaike Information Criterion of the model. After this variable was added, the pool of candidate variables was reduced to no longer include the variable that was just added to the model or any variables that were correlated with that variable with a correlation of greater than 0.5 or less than −0.5. The predicted odds generated by the multivariate model were converted into probabilities that an aneurysm would be symptomatic for each of the aneurysms in our data. Sensitivity, specificity, and negative predictive value measurements were also computed for all candidate predictor variables. Optimal cutoff points for continuous measures were found using Youden's J statistic.

### Results

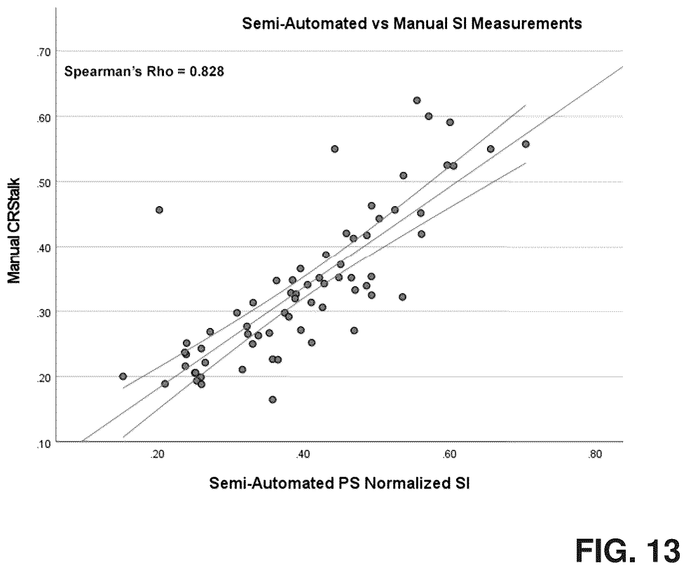

Demographics and Aneurysm Characteristics: A total of 233 patients underwent HR-MRI. One hundred sixty patients with fusiform, thrombosed, small (<2 mm) aneurysms, and poor quality/incomplete imaging were excluded. A total of 73 patients with 93 aneurysms (88 unruptured, 5 ruptured) were included. Demographic and morphological data are described in Table 8. A median (IQR) of 316 (421) SI probes were generated per aneurysm. The 3D-AWE method had a strong correlation with manual 2D measurements of SI (Spearman's rho=0.828) (FIG. 13).

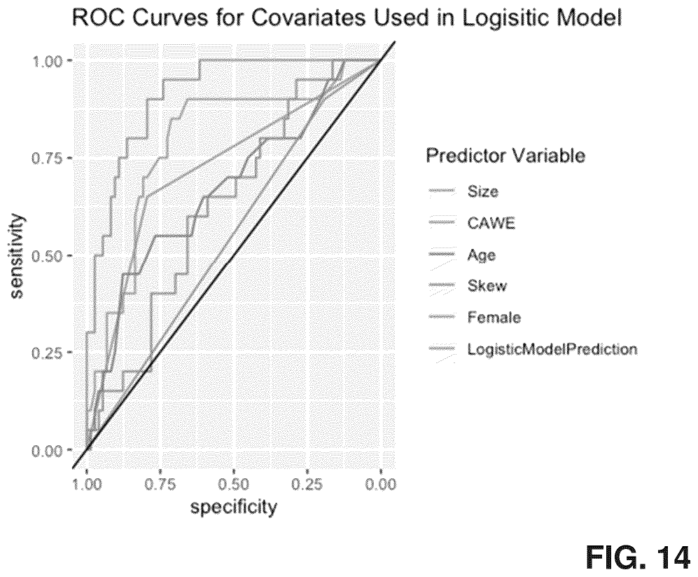

Aneurysm Wall Enhancement in Symptomatic Aneurysms: Age, aneurysm size, SR, AR, 3D-CAWE, SAWE, and FAWE (skewness) were independent predictors of symptomatic status. (Table 9, Table 10. In some aneurysms, the three enhancement metrics (3D-CAWE, SAWE, and FAWE) were useful in predicting symptomatic status regardless of aneurysm size (FIG. 12). Multivariate models that were fixed to include 3D-CAWE, SAWE, or FAWE as predictors were highly sensitive and specific. (Table 11. The best multivariate model, including age, size, 3D-CAWE+, skewness, and female covariates, had 80% specificity and 90% sensitivity in detecting symptomatic aneurysms (AUC=0.914, NPV=0.967). (FIG. 14.

Using the best model, the predicted probability of symptomatic status was calculated for each aneurysm. Fourteen (15%) of the aneurysms in the cohort had a higher than 50% probability of symptomatic status. Most of these aneurysms (71%) actually presented as symptomatic aneurysms. Fifty-one (55%) aneurysms had <10% probability of being symptomatic, and only one (2%) of these aneurysms, an 8 mm M2 MCA aneurysm, was actually symptomatic at presentation. These aneurysms were smaller in size [median (IQR)=4.6 (1.75) vs. 6.8 (4.6), p<0.001], had a lower size ratio [median (IQR)=2.17 (1.75) vs. 2.67 (2.26), p=0.02], and a lower AR [median (IQR)=1.35 (0.67) vs. 1.67 (1.51), p=0.03]. These aneurysms were also less enhancing, with a lower 3D-CAWE [mean±SD=0.80±0.24 vs. 0.97±0.27, p=0.003], lower SAWE [median (IQR)=0.16 (0.20) vs. 0.31 (0.39), p=0.002], and lower FAWE [mean±SD=0.32±0.74 vs. 0.19±0.57, p<0.001]. These aneurysms were also less likely to be present in smokers (p=0.027) and had lower ELAPSS scores (mean±SD=13±6 vs. 16±8, p=0.039).

Performance of AWE Metrics when analyzed with morphological data: Aneurysms ≥7 mm in diameter (n=64) had a higher 3D-CAWE [mean±SD=0.99±0.21 vs. 0.82±0.28, p=0.004] and higher SAWE [median(IQR)=0.30 (0.26) vs. 0.16 (0.27), p=0.006] than smaller aneurysms. Symptomatic aneurysms had a higher 3D-CAWE (mean±SD=1.03±0.25) than asymptomatic aneurysms (mean±SD=0.83±0.26, p=0.003). Most symptomatic aneurysms were 3D-CAWE+[13 symptomatic (65%) vs. 7 (35%) asymptomatic aneurysms (p<0.001)].

FAWE was higher in aneurysms with irregular morphology (n=34, mean±SD=0.11±0.55 vs. −0.20±0.78, p=0.043), high PHASES (≥5) scores (n=44, mean±SD=0.25±0.52 vs. −0.39±0.73, p<0.001), high ELAPSS (≥15) scores (n=44, mean±SD=0.10±0.54 vs. −0.25±0.81, p=0.018), and in symptomatic aneurysms (n=20, mean±SD=0.20±0.56 vs. −0.16±0.73, p=0.045). Aneurysms in high-risk locations (n=35) had a higher FAWE compared to aneurysms in other locations (mean±SD=0.22±0.61 vs. −0.27±0.72, p=0.001). This finding was consistent for smaller aneurysms (<7 mm) as well (mean±SD=0.29±0.69 vs. −0.39±0.75, p=0.001).

Bleb Analysis: Thirty-four aneurysms had irregular morphology; 21 (62%) had blebs and 4 (12%) were multilobulated. Approximately 61% of blebs enhanced more than the rest of the aneurysm. On average, these enhancing blebs had 17% higher SI and 56% more contrast uptake than the aneurysm sac (FIG. 13). In each of the four multilobulated aneurysms, one lobe showed increased SI compared to the rest of the aneurysm. These lobes had 11% higher SI and 65% more contrast uptake than the rest of the aneurysm.

Microhemorrhage Analysis: Only 46 unruptured aneurysms were analyzed with QSM due to image artifact. Six aneurysms were QSM+ and showed increased enhancement in all three metrics (3D-CAWE, SAWE, and FAWE). SAWE was significantly higher in QSM+ compared to QSM− aneurysms [median (IQR)=0.30 (0.23) vs. 0.13 (0.23), p=0.047] (FIG. 4).

### Discussion

AWE has consistently been associated with aneurysm instability. However, previous studies have been limited by 2D sampling of the aneurysm wall in multiplanar views. Herein it is demonstrated that 3D-AWE is highly predictive of symptomatic presentation. 3D-CAWE, SAWE, and FAWE had a strong association with known features of aneurysm instability. 3D mapping of AWE may be a powerful method of identifying both symptomatic and highly unstable asymptomatic aneurysms.

Advantages of 3D-AWE Mapping: The analysis of AWE has been done both subjectively and objectively using 2D multiplanar imaging. Finding objective methods to measure AWE of the entire aneurysm in 3D may enhance understanding of the possible association between AWE and the risk of aneurysm rupture.

The pipeline implemented in this analysis generates three strong AWE metrics that describe different biological processes. The first is 3D-CAWE, which is used to quantify AWE for the entire aneurysm relative to the corpus callosum based only on T1+Gd imaging. The second is SAWE, which compares the SI in T1 vs. T1+Gd images to determine the average level of contrast uptake for the entire aneurysm. And the third metric, FAWE, quantifies focal AWE, or regions of the aneurysm that are more enhancing than the rest of the aneurysm sac. This is measured using the skewness of the histogram on T1+Gd imaging. On top of having 3D color maps, the pipeline allows the generation of histograms that improve quality control and quantifies the distribution of enhancement along the aneurysm wall.

Aneurysm Wall Enhancement in Symptomatic Aneurysms: A prior systematic review and meta-analysis of >1000 aneurysms demonstrated a positive association between AWE and aneurysm rupture, growth, or symptomatic presentation. Another prior analysis of 341 aneurysms identified AWE as an independent risk factor associated with symptomatic aneurysms (defined by the presence of sentinel headache or oculomotor nerve palsy). This prior study used 2D multiplanar qualitative and quantitative assessments of AWE. Other studies have found a similar relationship between AWE and symptomatic presentation.

As discussed herein, AWE can provide insight into potential aneurysm instability. The multivariate model generated through our pipeline used AWE, age, aneurysm size, and female sex in detecting symptomatic aneurysms with an 80% specificity and 90% sensitivity (AUC=0.914, NPV=0.967). Other known factors associated with higher risk of aneurysm rupture, such as SR, AR, location, size, and high PHASES and ELAPSS scores, also had a strong correlation with every AWE metric generated through this pipeline.

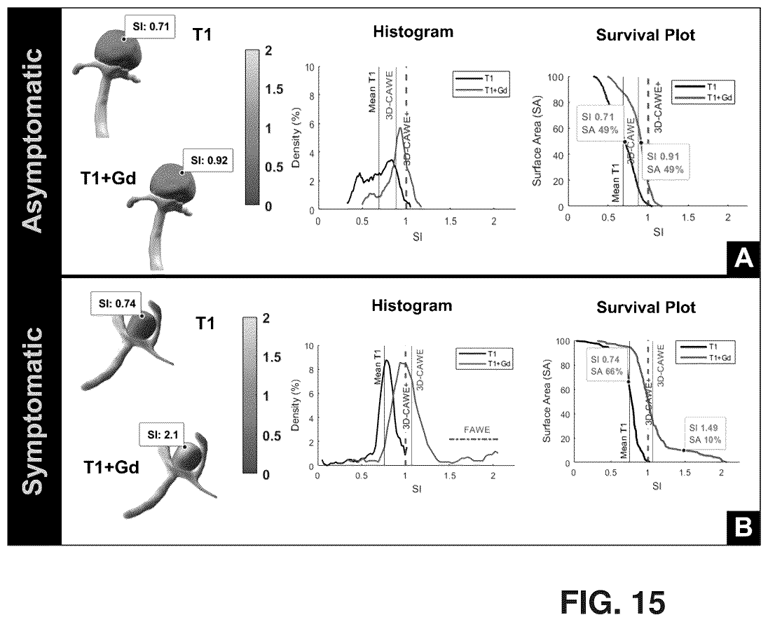

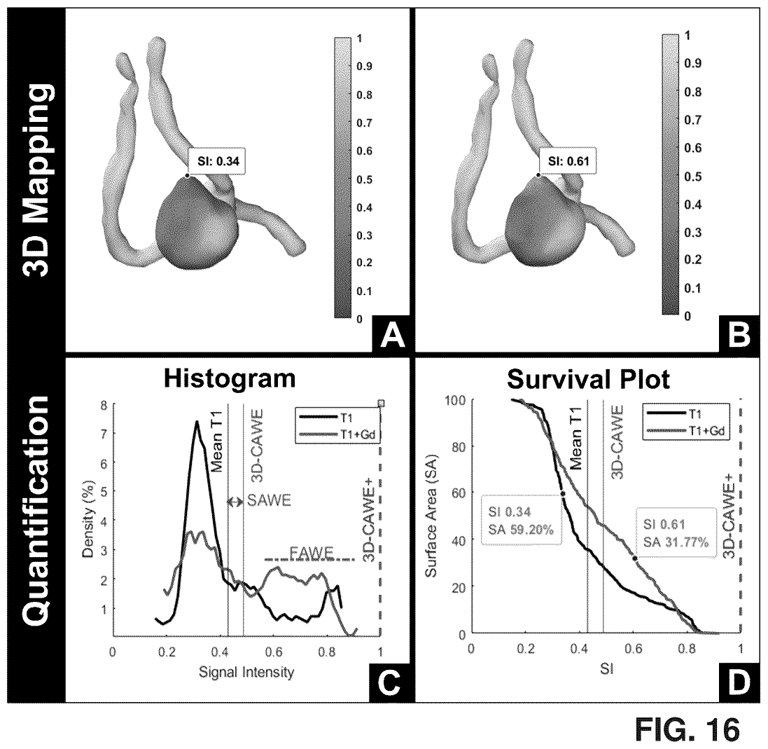

FAWE and Bleb Analysis: Irregular morphology is a risk factor of aneurysm rupture. In this Example, aneurysms with irregular morphology had increased FAWE (p=0.043). Small aneurysms in locations associated with a high risk of rupture also had increased FAWE (p=0.001). Better methods are required to identify small aneurysms that would not conventionally be deemed as high a risk, based on ISUIA criteria. Smaller aneurysms (<7 mm), such as aneurysms located in the anterior communicating artery, posterior communicating artery, and post inferior cerebellar artery, often present ruptured. In this Example, these aneurysms had larger areas of FAWE when compared to larger aneurysms (FIG. 15). 2D multiplanar studies have described the colocalization of FAWE with points of rupture. 3D-AWE mapping provides both quantitative and qualitative measures of such areas and might improve identification of these high-risk areas. On average, blebs took up 56% more contrast than the aneurysm sacs. (FIG. 16). The presence of blebs is a strong risk factor for aneurysm rupture. Compartmental analysis of contrast uptake allows for identifying focal areas of wall thinning and/or degradation such as blebs or areas prone to develop blebs.

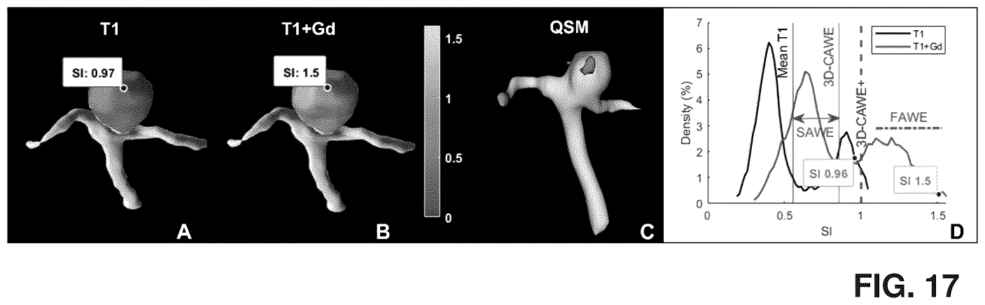

Microhemorrhage Analysis: In this Example, 46 aneurysms had appropriate QSM imaging for analysis. Six QSM+ aneurysms had significantly higher SAWE (p=0.047). Areas of microhemorrhage colocalized with increased AWE (FIG. 17). This highlights the potential of using SAWE as a metric for identifying areas with a high risk of rupture. Since SAWE determines Gd uptake by comparing the difference between T1+Gd and T1, it could be used to identify aneurysms with high-risk areas associated with microhemorrhages.

### Example 4

The analysis of atherosclerotic plaque enhancement with high resolution MRI (HR-MRI) after the administration of gadolinium (Gd) allows for the identification of symptomatic culprit plaques. Current HR-MRI methods are limited by 2D multiplanar views and manual sampling of regions of interest. Discussed herein is a semiautomated 3D method to objectively quantify enhancement of plaques and the parent artery.

### Methods

Patients diagnosed with stroke due to atherosclerotic plaques underwent 7T HR-MRI. Plaques in the vascular territory of the stroke were categorized as culprit. 3D segmentations of the plaque and parent artery were generated. Probes were then orthogonally extended from the arterial lumen into the plaque and the arterial wall. Signal intensity (SI) values were obtained and normalized to the corpus callosum (CC). Various measures of enhancement were analyzed through 3D color maps.

Image acquisition. After approval from the institutional review board, patients with stroke attributable to ICAD underwent 7T MRI after informed consent. ICAD stroke etiology was adjudicated based on TOAST and ASCOD criteria. Patients were excluded if they had any contraindication for 7T MRI, were medically unstable, or had a glomerular filtration rate <45 mL/min per 1.73 m². Images were obtained between August 2018 and July 2021.

HR-MRI was acquired with a GE MR950 7T scanner, using an 8-channel head coil (GE Healthcare, Waukesha, WI). 3D T1-weighted fast-spin-echo (CUBE) were acquired before and five minutes after the administration of Gd. Technical parameters for acquisition are described in the Table 12. Images were analyzed in at least three planes to identify the presence of atherosclerotic plaques using the Picture Archiving Communication System (Carestream Vue, version 12.1.6.1005). Plaque presence was defined as wall thickening of a vessel segment compared to the proximal and/or normal vessel segment. The following arterial segments were analyzed: supraclinoid internal carotid arteries, middle cerebral arteries, anterior cerebral arteries, V4 segments of the vertebral arteries, basilar artery, and posterior cerebral arteries in each subject to identify the presence of plaques. The specific stroke mechanism due to intercranial atherosclerotic disease was also adjudicated based on previously described criteria. WASID-based degree of stenosis, plaque burden, remodeling index, and area degree of stenosis were manually calculated in several planes as previously described.

Plaque Characterization. Plaques were classified as culprit vs. non-culprit after detection of the presence of plaques. In general, a culprit plaque was identified as a lesion in the ipsilateral vascular territory of an area of infarction accompanied by clinical symptoms. If more than one plaque was present in the same vascular territory, the most stenotic lesion was selected for analysis. Other plaques in the same vascular territory of culprit plaques were excluded from the analysis. Plaques located in other vascular territories were considered asymptomatic or non-culprit. The morphological characteristics and enhancement patterns of culprit plaques were compared with non-culprit plaques.

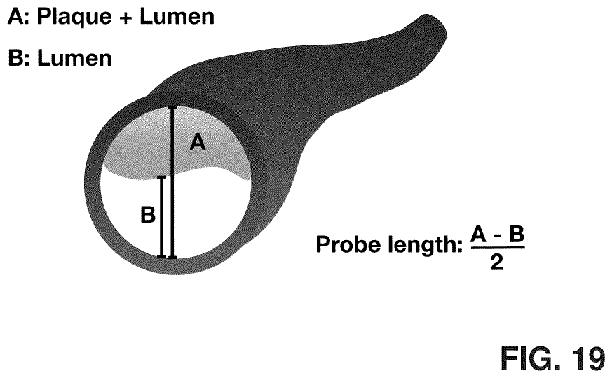

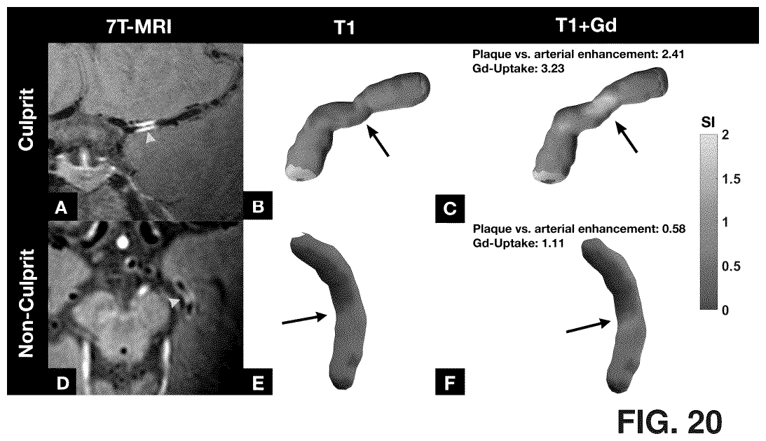

3D Plaque Analysis. The semiautomated method of analyzing the Gd enhancement of the wall of brain aneurysms was modified to study plaques and their parent arteries. Luminal segmentations in the vascular territory of the plaque were created manually using 3D Slicer on T1 and T1+Gd images. The arterial lumen was segmented from the center of the plaque and extending approximately 5 mm beyond the plaque boundaries, such that the plaque and the contiguous parent artery were included. Signal intensity (SI) probes were orthogonally extended from the lumen into the plaque and arterial wall (FIG. 18). The high spatial resolution of 7T imaging allowed a very accurate estimation of the arterial wall and plaque thickness. For the parent vessel analysis probes were extended 0.5 mm into the arterial wall of the parent vessel, which was the average wall thickness and has been reported by others in ex vivo samples. For the plaque analysis, the probe's length was customized based on detailed measurements of plaque thickness obtained on 7T MRI (FIG. 19). SI color maps were generated through an automated process using image processing tools and custom scripts in MATLAB 2020b (The MathWorks, Natick, MA). The SI of the plaque and arterial wall was mapped from T1 and T1+Gd images separately (FIG. 20). The SI of the genu of the corpus callosum (CC) was used to obtain a normalized mean enhancement ratio.

3D Analysis of Gd Enhancement. The highest SI of each probe on the T1 and T1+Gd was obtained for the analysis. The mean (m) SI and standard deviation (SD) of every plaque and parent artery were calculated. These values were then divided by the SI of the CC to obtain a normalized ratio that could be used to compare among subjects. The following metrics of enhancement were studied:
1) The mean enhancement ratio=mean SI on T1+Gd normalized to the CC. This is the standard measurement of contrast enhancement.
2) Plaque enhancement variance=SD² of the SI values measured by the probes on T1+Gd. This measurement was used to analyze the dispersion of enhancement within the plaque.
3) Gd-uptake, which measured the amount of Gd absorbed by the plaque when compared to T1:

$$Gd uptake = \frac{{μ}_{P l aque T 1 + G d} - {μ}_{P l aque T 1}}{S {D}_{P l aque T 1}}$$

Previous examples have shown that atherosclerotic plaques have a wide dispersion of SI with highly variable SDs. To account for this SI variability, SD was used as a normalization factor in Gd-uptake.
4) Plaque vs arterial enhancement, which measured the difference in the mean enhancement of the plaque compared to the parent artery.

$$Plaque vs arterial enhancement = \frac{{μ}_{P l aque T 1 + G d} - {μ}_{V e ssel T 1 + G d}}{S {D}_{Vessel T 1 + G d}}$$

The SD of the arterial SI was used for normalization and to perform standardized comparisons of plaques in different territories.

Statistical analysis. Various statistical analyses were conducted using SPSS Statistics 27.0 (IBM, Armonk, New York). Categorical variables are presented as frequency and percent, continuous variables are described as mean±SD. Shapiro-Wilk tests were used to evaluate normality for variables of interest within our sample, with p-values <0.05 being considered non-normally distributed variables. A Student's t-test was used to compare the means between normally distributed variables and a Mann Whitney U test was used to compare the distributions between non-normally distributed variables. Chi-square tests were used for comparing categorical variables. Univariate logistic regressions were performed to analyze relevant demographic, morphologic and signal intensity variables. An alpha threshold of 0.05 was used to assess significance for all hypothesis tests.

The multivariate model in this analysis was constructed through an Akaike information criterion-based forward selection procedure. All variables of interest in the dataset were initially considered candidates for the multivariate model, and the variable whose addition most reduced the Akaike information criterion of the model was added to the model. For the addition of further variables in the model, variables were only considered candidates for inclusion into the model if they had a correlation <0.5 with each variable that was already added to the model. Once there were no remaining variables left that reduced the Akaike information criterion value of the multivariate model and had correlations <0.5 with each variable present in the multivariate model, the algorithm was terminated, and the resulting model was selected.

Receiver operating characteristic metrics including sensitivity and specificity were calculated for each variable of interest (including the predictions output from the multivariable model) at its optimal threshold value, as determined by Youden's J statistic. Areas under the curve values for each variable were also computed.

### Results

Thirty-six culprit and 44 non-culprit plaques from 36 patients were analyzed. Culprit plaques had higher mean enhancement ratios (P=0.004) and Gd-uptake (p<0.001) than non-culprit plaques. Enhancement was more heterogeneous in culprit plaques (p=0.041). Gd-uptake was the most accurate metric for predicting culprit plaques (OR 3.9; Cl 2.1-8.3). When combining Gd-uptake and plaque burden, a sensitivity of 83% and a specificity of 86% was achieved for predicting culprit plaque presence.

Patients and plaque characteristics. Thirty-six patients with 80 plaques were included in the analysis. Approximately 47.2% were men and the mean age was 59±12 years. The mean National Institute of Health Stroke Scale at admission was 2.5±3. Additional demographics are described in Table 13. Culprit plaques had a higher degree of stenosis, plaque burden, and remodeling index than non-culprit plaques. In addition, culprit plaques more commonly had a concentric morphology than non-culprit plaques (Table 14).

3D Enhancement Characterization. A mean of 125±79 probes/plaque was generated. Culprit plaques had a higher mean enhancement ratio than non-culprit plaques (μ=0.80±0.30 vs. 0.61±0.17; P=0.004). This enhancement was more heterogenous in culprit plaques (higher variance) than non-culprit plaques (SD²=0.33±0.13, vs. 0.26±0.08; p=0.041). Culprit plaques took up significantly more Gd than non-culprit plaques (2.01±0.91 vs. 1.06±0.76; p<0.001) (FIG. 20).

In univariate logistic regressions several metrics were predictors of culprit plaques (Table 15). The following variables were used to generate a multivariate logistic regression model of culprit plaque prediction: plaque burden (odds ratio 3.7, 95% confidence interval 1.6-10; p=0.004), Gd-uptake (odds ratio 2.3, 95% confidence interval 1.1-5.3; p=0.033), and stenosis (odds ratio 1.3, 95% confidence interval 0.7-2.7; p=0.4). The combined receiver operating characteristic analysis of this model had a sensitivity of 83% and a specificity of 86% (area under the curve=0.87).

A previous method described that a CRₛₜₐₗₖ of ≥0.53 had a sensitivity of 78% and a specificity of 62% in detecting culprit plaques (area under the curve=0.76). In contrast, using this new 3D method, a Gd-uptake threshold of ≥1.23 had a sensitivity of 86% and a specificity of 71% (area under the curve=0.81).

Parent arteries of Culprit and Non-culprit plaques. A mean of 858±564 probes/arterial segment was generated. There was no statistical difference of enhancement between parent arteries of culprit and non-culprit plaques (m=0.59±0.20 vs. 0.62±0.14; p=0.62).

### Discussion

The identification of symptomatic plaques will improve the diagnosis of intracranial atherosclerotic disease as stroke etiology and may guide targeted endovascular interventions such as angioplasty and stenting. In this disclosure, a method to quantify Gd enhancement, which generates 3D enhancement maps from hundreds of datapoints. Several enhancement metrics generated through this method enable the detailed analysis of plaque and its parent artery enhancement. This method is more accurate in detecting and quantifying Gd enhancement than the conventional 2D multiplanar method.

Gd-uptake was the most accurate enhancement metric in detecting culprit plaques (odds ratio 3.9, confidence interval 2.1-8.3). This metric quantifies the difference of enhancement between T1 and T1+Gd. It is similar to “enhancement ratio”, which has been shown in other studies as an independent predictor of stroke in patients with intracranial atherosclerotic disease. One key difference is that Gd-uptake accounts for the inherent heterogenicity of enhancement on T1. This metric is aimed at analyzing plaques individually. Mean plaque enhancement ratio, plaque vs. arterial enhancement and plaque enhancement variance were also significant in detecting culprit plaques. This heterogeneity in the distribution of enhancement can be seen in the 3D color maps of the plaque and parent vessel (FIGS. 18 and 20). Subtle color differences along the diseased arterial segment reflect different enhancement patterns, which ultimately suggests different biological processes and stages of disease progression. Moreover, the detailed quantification of Gd enhancement has the potential of determining the response to medical therapy.

3D methods of vessel analysis have been used previously to visualize morphological features such as vessel caliber and angulation. The analysis of vessel wall geometries has been shown to influence the effectiveness of endovascular interventions. Volumetric 3D maps of intracranial atherosclerotic disease allow a more detailed characterization of plaque burden, remodeling index, and subtle differences in plaque morphology within different vascular territories. 3D analysis allows an objective survey of enhancement of the target arterial segment. The method may generate vessel wall metrics not limited to Gd enhancement. The quantification of enhancement through 3D analysis allows for easier identification of potential culprit plaques.

The identification of symptomatic plaques through HR-MRI could lead to the diagnosis of stroke etiology. A review showed that over half of patients without significant luminal stenosis might have a culprit intracranial plaque identified on HR-MRI. Detailed analysis of the vessel segments through the generation of 3D enhancement color maps could increase the detection of atherosclerotic changes in patients with cryptogenic stroke. The combined objective quantification of Gd uptake with plaque burden achieved a sensitivity of 83% and specificity of 86% in detecting culprit plaques. Moreover, when this 3D method was compared with previous work that use 2D measurements of enhancement, it proved to be more sensitive and specific.

This method is more accurate than the standard methods that rely on 2D multiplanar analysis in detecting culprit plaques.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

### ACRONYMS

AWE—aneurysmal wall enhancement
GAWE—generalized aneurysmal wall enhancement
SAWE—specific aneurysmal wall enhancement
FAWE—focal aneurysmal wall enhancement
CAWE—circumferential aneurysmal wall enhancement
Gd—gadolinium
CC—corpus collosum
SI—signal intensity
QSM—quantitative sustainability mapping
SD—standard deviation
HR-MRI—high resolution-magnetic resonance imaging
CRₛₜₐₗₖ—contrast ratio normalized to the pituitary stalk
ACOM—anterior communicating artery
PCOM—posterior communicating artery
PICA—post inferior cerebellar artery

Although the disclosure has been described with reference to preferred embodiments, persons skilled in the art will recognize that changes may be made in form and detail without departing from the spirit and scope of the disclosed apparatus, systems and methods.

TABLE 1
Characteristics of saccular aneurysms.

| | | | | |
|---|---|---|---|---|
| | Total Aneurysms | GAWE (+) | GAWE (−) | |
| Variable | N = 28 | N = 7 | N = 21 | P |

Demographic

| | | | | |
|---|---|---|---|---|
| Age(mean ± SD, years) | 66 ± 9.1 | 72 ± 8.8 | 64 ± 8.5 | 0.04 |
| Men(%) | 8(29) | 1(14) | 7(33) | 0.33 |
| Hypertension(%) | 18(64) | 6(86) | 12(57) | 0.17 |
| Jyperlipidemia(%) | 13(46) | 3(43) | 10(48) | 0.83 |
| Smoking(%) | 6(21) | 0 | 6(29) | 0.11 |

Morphologic

| | | | | |
|---|---|---|---|---|
| Diameter (mean ± SD, mm) | 8.2 ± 6.56 | 12.39 ± 8.13 | 6.8 ± 5.5 | .049 |
| Size Ratio (mean ± SD) | 2.51 ± 1.78 | 3.95 ± 2.59 | 2.02 ± 1.12 | 0.010 |
| Aspect Ratio (mean ± SD) | 2.12 ± 1.61 | 3.63 ± 2.35 | 1.61 ± 0.89 | 0.002 |
| Ellipticity Index (mean ± SD) | 0.24 ± 0.03 | 0.24 ± 0.02 | 0.22 ± 0.03 | 0.2 |
| Non-Sphericity Index | 0.28 ± 0.05 | 0.31 ± 0.04 | 0.27 ± 0.05 | 0.13 |
| (mean ± SD) | | | | |
| Undulation Index (mean ± SD) | 0.15 ± 0.06 | 0.16 ± 0.06 | 0.14 ± 0.6 | 0.48 |
| Surface Area | 311.8 ± 680.4 | 617.6 ± 852.5 | 209.8 ± 602.7 | 0.17 |
| (mean ± SD, mm²) | | | | |
| Volume (mean ± SD, mm³) | 913 ± 2729.1 | 1769.5 ± 3198.6 | 627.5 ± 2577.4 | 0.35 |
| Wall Thickness | 0.55 ± 0.09 | 0.62 ± 0.1 | 0.51 ± .09 | 0.008 |
| (mean ± SD, mm) | | | | |
| Bleb (%) | 14(50) | 3(43) | 11(52) | 0.66 |
| Mural Thrombosis | 4(14) | 3(43) | 1(4) | 0.013 |

Location

| | | | | |
|---|---|---|---|---|
| ICA (%) | 12(43) | 3(43) | 9(43) | 0.163 |
| ACOM (%) | 7(25) | 1(14) | 6(29) | |
| Basilar (%) | 3(11) | 1(14) | 2(10) | |
| MCA (%) | 4(14) | 0 | 4(19) | |
| PICA (%) | 1(4) | 1(14) | 0 | |
| PCOM (%) | 1(4) | 1(14) | 0 | |

Aneurysm Wall Enhancement (AWE)

| | | | | |
|---|---|---|---|---|
| Δ CC Ratio (mean ± SD)* | 0.85 ± 0.24 | 0.56 ± 0.15 | 0.24 ± 0.12 | 0.001 |
| Percent Increase (mean ± SD)* | 63 ± 41 | 96 ± 48 | 52 ± 0.33 | 0.012 |

*T1 to T1 + Gd

TABLE 2
Distribution of enhancement ratios of the PS and CC for aneurysms and blebs

| | | T1 | | T1 + Gd | | | |
|---|---|---|---|---|---|---|---|
| | | | Coefficient | | Coefficient of | % Change from | |
| | | Mean ± SD | of Variation | Mean ± SD | Variation | Pre to Post | |
| Aneurysm | Pituitary | 0.482 ± 0.178 | 36.89% | 0.424 ± 0.159 | 37.73% | −12.04% | (p = 0.1) |
| | Stalk | | | | | | |
| | Corpus | 0.564 ± 0.165 | 29.18% | 0.892 ± 0.25 | 28.06% | 58% | (p < 0.0001) |
| | Callosum | | | | | | |
| Bleb | Pituitary | 0.474 ± 0.152 | 32.06% | 0.427 ± 0.202 | 47.2% | −9.92% | (p = 0.28) |
| | Stalk | | | | | | |
| | Corpus | 0.548 ± 0.151 | 27.6% | 0.905 ± 0.319 | 35.28% | 65.15% | (p < 0.0001) |

Callosum

TABLE 3
Technical parameters for HR-VWI acquisition
in a 7T GE MR950 MRI Scanner.

| | Image Sequence | | |
|---|---|---|---|
| Parameter | T1 CUBE | T2 CUBE | 3D TOF |
| TR (msec) | 700 | 2500 | 40 |
| TE (msec) | 10.3 | 71.84 | 2.3 |
| | (minimum) | | |
| Flip angle | 90 | 90 | 12 |
| Bandwidth (Hz/pixel) | 62.5 | 62.5 | 62.5 |
| FOV (mm) | 512 × 512 | 256 × 256 | 512 × 512 |
| Matrix (mm) | 256 × 256 | 256 × 256 | 512 × 320 |
| Voxel size (mm) | 0.5 × 0.5 × 1 | 1 × 1 × 1 | 0.43 × 0.43 × 1.0 |
| Slice thickness (mm) | 0.5 | 1.0 | 1.0 |
| ETL | 22 | 100 | 1 |

CE-MRA: contrast-enhanced MRA;
ETL: echo train length;
FOV: field of view;
TE: echo time;
TR: response time.

TABLE 4
Demographic and location data for all aneurysms in cohort.

| | | Size | Size | Aspect | | | | Current |
|---|---|---|---|---|---|---|---|---|
| Location | Shape | (mm) | Ratio | Ratio | Sex | Age | HTN | Smoker |
| PICA | Saccular | 3.8 | 1.24 | 1.75 | F | 82 | Y | N |
| ACOM | Saccular | 5.9 | 1.68 | 1.29 | M | 57 | N | N |
| ACOM | Saccular | 3.2 | 1.25 | 2.00 | M | 77 | N | N |
| ICA | Saccular | 16.7 | 7.52 | 6.35 | F | 61 | N | N |
| ACOM | Saccular | 6.4 | 2.89 | 1.16 | M | 77 | Y | N |
| ACOM | Saccular | 4.4 | 1.65 | 1.07 | M | 71 | Y | Y |
| BA | Saccular | 3.8 | 1.87 | 1.67 | F | 69 | Y | N |
| ICA | Saccular | 3.9 | 1.09 | 1.44 | F | 63 | Y | N |
| PCOM | Saccular | 16.8 | 3.57 | 5.29 | F | 69 | Y | N |
| ICA | Saccular | 5.9 | 0.68 | 0.95 | F | 73 | Y | Y |
| ICA | Saccular | 4.1 | 0.69 | 1.06 | F | 76 | Y | N |
| ICA | Saccular | 2.8 | 0.72 | 1.27 | F | 64 | N | Y |
| BA | Saccular | 4.8 | 2.61 | 1.09 | F | 64 | N | Y |
| ICA | Saccular | 5.9 | 1.05 | 0.93 | F | 71 | N | N |
| ICA | Saccular | 10.2 | 3.27 | 2.87 | F | 61 | N | N |
| MCA | Saccular | 3.5 | 1.11 | 1.27 | F | 79 | Y | N |
| MCA | Saccular | 7.8 | 3.52 | 0.91 | M | 68 | Y | Y |
| ICA | Saccular | 3.6 | 0.85 | 1.03 | F | 63 | N | N |
| ACOM | Saccular | 8.2 | 3.78 | 2.32 | M | 61 | Y | N |
| BA | Saccular | 13.1 | 6.48 | 6.24 | F | 60 | Y | N |
| MCA | Saccular | 7.0 | 2.16 | 0.69 | M | 51 | Y | Y |
| ACOM | Saccular | 5.3 | 1.37 | 1.60 | F | 62 | Y | N |
| MCA | Saccular | 7.1 | 2.46 | 2.37 | M | 58 | Y | N |
| ACA | Saccular | 7.7 | 3.29 | 2.31 | F | 74 | Y | N |
| BA | Fusiform | 11.3 | N/A | N/A | F | 68 | Y | N |
| BA | Fusiform | 11.3 | N/A | N/A | F | 68 | Y | N |
| ICA | Fusiform | 5.3 | N/A | N/A | M | 62 | N | N |
| BA | Fusiform* | 27.0 | N/A | N/A | M | 59 | Y | Y |
| ICA | Fusiform | 25.8 | N/A | N/A | F | 79 | Y | N |
| ICA | Fusiform | 5.9 | N/A | N/A | F | 49 | N | N |
| ICA | Fusiform | 6.7 | N/A | N/A | F | 49 | N | N |
| ICA | Fusiform* | 29.3 | N/A | N/A | F | 60 | Y | N |

*Indicates presence of intramural thrombosis.
HTN = hypertension

TABLE 5
Morphological characteristics of saccular
aneurysms with different enhancement patterns.

| | | Size (mm) | Aspect Ratio | Size Ratio |
|---|---|---|---|---|
| General Enhancement | GAWE+ (N = 6) | 10.15 ± 6.11 | 3.73 ± 2.76 | 3.64 ± 2.57 |
| (N = 24) | GAWE− (N = 18) | 5.61 ± 2.05 | 1.91 ± 1.02 | 1.51 ± 0.62 |
| | p | 0.137 | 0.199 | 0.104 |
| Specific Enhancement | SAWE+ (N = 11) | 7.96 ± 5.16 | 2.79 ± 2.36 | 2.64 ± 2.17 |
| (N = 24) | SAWE− (N = 13) | 5.71 ± 2.12 | 2.01 ± 0.96 | 1.52 ± 0.68 |
| | p | 0.424 | 0.776 | 0.277 |
| Combined Enhancement | GAWE+/SAWE+ (N = 6) | 10.15 ± 6.11 | 3.73 ± 2.76 | 3.64 ± 2.57 |
| (N = 24) | GAWE−/SAWE− (N = 13) | 5.71 ± 2.12 | 2.01 ± 0.96 | 1.53 ± 0.67 |
| | p | 0.179 | 0.244 | 0.106 |

*GAWE = General Aneurysm Wall Enhancement,
SAWE = Specific Aneurysm Wall Enhancement

TABLE 6
AWE of saccular and fusiform aneurysms.

| | Saccular (N = 24) | Fusiform (N = 8) | p |
|---|---|---|---|
| Size (mm)^{†} | 6.75 ± 3.91 | 15.3 ± 10.3 | 0.015 |
| Wall Thickness (mm) | 0.527 ± 0.093 | 0.603 ± 0.083 | 0.049 |
| Circumferential SI (μₚₒₛₜ) | 0.846 ± 0.246 | 1.005 ± 0.1937 | 0.108 |
| % Increase in Circumferential | 61.89 ± 41.13 | 57.47 ± 35,95 | 0.931 |
| SI^{†}* | | | |
| Number of Focal Areas of | 1.083 ± 0.829 | 3.378 ± 2.386 | <0.001 |
| Enhancement^{†} | | | |
| Area of FAWE (%)^{†} | 1.82 ± 1.65 | 4.09 ± 1.07 | 0.003 |
| Coefficient of Variation (%)^{†} | 19.5 ± 7.38 | 24.8 ± 7.26 | 0.045 |
| Skewness^{†} | −0.337 ± 0.881 | 0.591 ± 0.4225 | 0.003 |
| Kurtosis^{†} | 4.39 ± 2.86 | 3.879 ± 1.207 | 0.695 |
| SAWE Cutoff | 0.8204 ± 0.181 | 1.0974 ± 0.3448 | 0.006 |

^{†}Mann-Whitney U significance reported
*% Increase measured from T1 to T1 + Gd

TABLE 7
Imaging parameters for a 3T Siemens
MAGNETOM Skyra MRI scanner.

| | SAG 3D T1 | | | |
|---|---|---|---|---|
| | SPACE FS | CE-MRA | Ax 2D TOF | QSM |
| TR (msec) | 900 | 3.32 | 19 | 61 |
| TE (msec) | 15 | 1.28 | 4.2 | 7-56 |
| Flip angle (deg) | variable | 25 | 35 | 17 |
| Bandwidth (Hz/pixel) | 446 | 590 | 219 | 300-260 |
| FOV (mm) | 200 × 200 | 195 × 195 | 220 × 220 | 172 × 172 |
| Matrix (mm) | 320 × 320 | 288 × 288 | 256 × 256 | 218 × 218 |
| Voxel size (mm) | 0.6 × 0.6 × 0.6 | 0.6 × 0.6 × 0.8 | 0.9 × 0.9 × 3.0 | 0.4 × 0.4 × 1.5 |
| Slice thickness (mm) | 0.63 | 0.8 | 3.0 | 1.5 |
| Turbo Factor | 52 | — | — | — |
| Acquisition time (min) | 3:29 | 0:15 | 1:24 | 5:00 |

CE-MRA: contrast-enhanced MRA;
FOV: field of view;
TE: echo time;
TR: repetition time.

TABLE 8
Demographic data and
morphological measurements.
Demographics (n = 73)

| | |
|---|---|
| Women | 60 (82%) |
| Smokers | 21 (29%) |
| Diabetes Mellitus | 9 (12%) |
| Hypertension | 45 (62%) |
| Polycystic Kidney Disease | 3 (4%) |
| History of SAH | 1 (1%) |
| Family History of IA | 7 (10%) |
| Multiple Aneurysms | 18 (25%) |

Aneurysms

| | Entire Cohort | Asymptomatic | Symptomatic | |
|---|---|---|---|---|
| | (n = 93) | (n = 73) | (n = 20) | p |
| Location | | | | |
| ICA | 31 (33%) | 22 (30%) | 9 (45%) | N/A |
| MCA | 21 (22%) | 18 (25%) | 3 (15%) | |
| ACOM | 14 (15%) | 10 (14%) | 4 (20%) | |
| BA | 9 (10%) | 7 (10%) | 2 (10%) | |
| PCOM | 8 (9%) | 7 (10%) | 1 (5%) | |
| ACA | 7 (8%) | 6 (8%) | 1 (5%) | |
| PICA | 1 (1%) | 1 (1%) | 0 (0%) | |
| SCA | 1 (1%) | 1 (1%) | 0 (0%) | |
| PCA | 1 (1%) | 1 (1%) | 0 (0%) | |
| Size | 5.25 (3.55) | 4.70 (2.90) | 7.95 (2.95) | <0.001 |
| Size Ratio | 2.33 (1.64) | 2.20 (1.69) | 3.80 ± 1.74 | <0.001 |
| Aspect Ratio | 1.52 (0.81) | 1.38 (0.76) | 2.03 (1.18) | <0.001 |
| Irregular | 34 (37%) | 23 (32%) | 11 (55%) | 0.053 |
| PHASES | 4 (4) | 4.00 (4.00) | 5.55 ± 3.03 | 0.049 |
| ELAPSS | 14.73 ± 7.01 | 13.66.82 | 18.7 ± 6.50 | 0.003^{†} |

*Categorical variables represented as frequency (percentage).
Normally distributed continuous variables are represented as mean ± SD.
Non-normally distributed continuous variables are represented as median (IQR).
^{†}Student's t-test significance reported
SAH = subarachnoid hemorrhage

TABLE 9
Logistic analysis of predictors of symptomatic status.

| | | | | | | | |
|---|---|---|---|---|---|---|---|
| OR | 95% CI | p | Cutoff | SP | SN | AUC | NPV |

Univariate Analysis

| | | | | | | | | |
|---|---|---|---|---|---|---|---|---|
| Variable | | | | | | | | |
| Female | 2.14 | 0.53-14.4 | 0.3 | 0.50 | 0.19 | 0.90 | 0.546 | 0.875 |
| HTN | 0.68 | 0.25-1.88 | 0.4 | 0.50 | 0.64 | 0.45 | 0.547 | 0.810 |
| Family Hx IA | 1.05 | 0.15-4.80 | >0.9 | 0.50 | 0.63 | 0.40 | 0.515 | 0.793 |
| Smoking | 1.90 | 0.68-5.24 | 0.2 | 0.50 | 0.90 | 0.10 | 0.502 | 0.786 |
| High Risk Location | 1.14 | 0.40-3.10 | 0.8 | 0.50 | 0.63 | 0.40 | 0.515 | 0.793 |
| Irregular | 2.66 | 0.97-7.47 | 0.058 | 0.50 | 0.69 | 0.55 | 0.617 | 0.847 |
| Age | 0.94 | 0.89-0.99 | 0.015 | 55.5 | 0.88 | 0.45 | 0.328 | 1.00 |
| Size | 1.53 | 1.23-1.98 | <0.001 | 6.2 | 0.71 | 0.85 | 0.790 | 0.945 |
| SR | 1.96 | 1.38-2.94 | <0.001 | 2.28 | 0.56 | 0.85 | 0.756 | 0.932 |
| AR | 2.09 | 1.25-3.80 | 0.008 | 1.94 | 0.86 | 0.55 | 0.750 | 0.875 |
| 3D-CAWE*† | 1.34 | 1.10-1.67 | 0.005 | 1.02 | 0.81 | 0.65 | 0.731 | 0.894 |
| SAWE* | 1.25 | 1.00-1.18 | 0.047 | 0.31 | 0.74 | 0.60 | 0.637 | 0.871 |
| Skew* | 1.08 | 1.00-1.18 | 0.049 | 0.20 | 0.66 | 0.60 | 0.629 | 0.857 |
| 3D-CAWE+† | 7.18 | 2.51-22.3 | <0.001 | 0.50 | 0.80 | 0.65 | 0.722 | 0.892 |

Multivariate Model

| | | | | | | | | |
|---|---|---|---|---|---|---|---|---|
| Covariates | | | | | | | | |
| Size | 1.49 | 1.18-2.01 | 0.003 | 6.2 | 0.71 | 0.85 | 0.790 | 0.945 |
| 3D-CAWE+ | 4.92 | 1.22-21.0 | 0.026 | 0.50 | 0.80 | 0.65 | 0,722 | 0.892 |
| Age | 0.89 | 0.81-0.96 | 0.007 | 55.5 | 0.88 | 0.45 | 0.328 | 1.00 |
| Skew* | 1.15 | 1.01-1.34 | 0.049 | 0.20 | 0.66 | 0.60 | 0.629 | 0.857 |
| Female | 6.05 | 0.78-123 | 0.14 | 0.50 | 0.19 | 0.90 | 0.546 | 0.875 |

| | | | | | |
|---|---|---|---|---|---|
| Model | N/A | 0.80 | 0.90 | 0.914 | 0,967 |

*Indicates OR calculated for a 0.1 unit increase, rather than 1.
†3D-CAWE is continuos, while 3D-CAWE+ is the dichotomized classification of aneurysms more enhancing than the corpus callosum (3D-CAWE ≥ 1)
HTN = hypertension,
Hx = history,
IA = intracranial aneurysm,
CAWE = circumferential aneurysm wall enhancement,
SAWE = specific aneurysm wall enhancement,
OR = odds ratio,
CI = confidence interval,
SP = specificity,
SN = sensitivity,
AUC = area under curve,
NPV = negative predictive value

TABLE 10
Pearson correlations between predictors
of symptomatic presentation

| | | | Size | Aspect | | |
|---|---|---|---|---|---|---|
| | Age | Size | Ratio | Ratio | 3D-CAWE | SAWE |
| Size | 0.010 | | | | | |
| Size Ratio | −0.003 | 0.757 | | | | |
| Aspect Ratio | −0.113 | 0.503 | 0.627 | | | |
| 3D-CAWE | −0.042 | 0.362 | 0.101 | 0.046 | | |
| SAWE | 0.073 | 0.242 | −0.076 | −0.022 | 0.840 | |
| Skew | 0.142 | 0.200 | 0.142 | 0.115 | 0.246 | 0.175 |

3D-CAWE: three-dimensionalcircumferential aneurysm wall enhancement,
SAWE: specific aneurysm wall enhancement

TABLE 11
Multivariate models using the three
enhancement metrics (3D-CAWE, SAWE, skew/FAWE)
to predict symptomatic presentation. Model 3 was
chosen as the best model.

| Variable | OR | 95% CI | p | Specificity | Sensitivity |
|---|---|---|---|---|---|
| Model 1 | | | | 85% | 83.6% |
| 3D-CAWE | 1.46 | 1.12-1.98 | 0.009 | | |
| Size Ratio | 2.05 | 1.37-3.33 | 0.001 | | |
| Age | 0.88 | 0.80-0.95 | 0.002 | | |
| Skew | 1.16 | 1.04-1.33 | 0.018 | | |
| Model 2 | | | | 90% | 75.3% |
| SAWE | 1.43 | 1.03-2.01 | 0.031 | | |
| Size Ratio | 2.36 | 1.56-3.91 | <0.001 | | |
| Age | 0.87 | 0.79-0.94 | 0.002 | | |
| Skew | 1.16 | 1.02-1.34 | 0.029 | | |
| Female | 5.68 | 0.81-66.8 | 0.11 | | |
| Model 3 | | | | 90% | 79.5% |
| Size | 1.15 | 1.18-2.01 | 0.003 | | |
| CAWE+ | 1.49 | 1.22-21.0 | 0.026 | | |
| Age | 0.89 | 0.81-0.96 | 0.007 | | |
| Skew | 4.92 | 1.01-1.34 | 0.049 | | |
| Female | 6.05 | 0.78-123 | 0.14 | | |

3D-CAWE: three-dimensional circumferential aneurysm wall enhancement,
SAWE: specific aneurysm wall enhancement,
FAWE: focal aneurysm wall enhancement

TABLE 12
Technical parameters
for HR-VWI acquisition in a 7T
GE MR950 7T scanner

| Parameter | 3D T1 SPACE |
|---|---|
| TR (msec) | 1400 |
| TE (msec) | 15.475 |
| Flip angle | 90 |
| Bandwidth (Hz/pixel) | 62.5 |
| FOV (mm) | 180 |
| Matrix (mm) | 384 × 384 |
| Voxel size (mm) | 0.5 × 0.5 × 0.5 |
| Slice thickness (mm) | 0.5 |
| ETL | 72 |
| Acquisition time | 7:16 |

ETL: echo train length;
FOV: field of view;
TE: echo time;
TR: response time.

TABLE 13
Demographic Data

| Demographic Data | |
|---|---|
| Men % (N) | 47.2 (17) |
| Diabetes Mellitus % (N) | 22.2 (8) |
| Hypertension % (N) | 72.2 (26) |
| Smoking % (N) | 47.2 (17) |
| Age (m ± SD) | 59 ± 12 |
| NIHSS at admission (m ± | 2.5 ± 3 |
| SD) | |
| Time from stroke to imaging | 4.25 ± 7.54 |
| (months) (m ± SD) | |
| Stroke Mechanism of the | |
| Culprit Atherosclerotic | |
| Plaque % (N) | |
| Athero-embolic | 30.5 (11) |
| Hypoperfusion | 33.3 (12) |
| Perforator-occlusion | 19.4 (7) |
| Unable to determinate | 16.6 (6) |

TABLE 14
Plaque characteristics.
Plaque Characteristics % (N)

| Plaques | Culprit (N = 36) | Non-Culprit (N = 44) | |
|---|---|---|---|
| MCA | 50 (18) | 40.9 (18) | |
| ICA | 13.9 (5) | 20.5 (9) | |
| ACA | 0 | 13.6 (6) | |
| Basilar | 19.4 (7) | 4.5 (2) | |
| PCA | 2.8 (1) | 20.5 (9) | |
| VA | 13.9 (5) | 0 (0) | |
| Morphology | | | P Values |
| Concentric % (N) | 72 (26) | 20 (9) | <0.001* |
| Stenosis % (mean ± | 46 ± 21 | 31 ± 15 | 0.001* |
| SD) | | | |
| PB % (mean ± SD) | 85 ± 13 | 65 ± 14 | <0.001* |
| RI (mean ± SD) | 0.37 ± 0.27 | 0.65 ± 0.22 | <0.001* |
| Area of stenosis % | 64 ± 24 | 35 ± 22 | <0.001* |
| K(mean ± SD) | | | |

Enhancement (mean ± SD)

| | | | |
|---|---|---|---|
| Mean enhancement | 0.80 ± 0.30 | 0.61 ± 0.17 | 0.004* |
| ratio plaque^{†} | | | |
| Plaque enhancement | 0.33 ± 0.13 | 0.26 ± 0.08 | 0.041* |
| variance | | | |
| Gd-uptake | 2.01 ± 0.91 | 1.06 ± 0.76 | <0.001* |
| Mean enhancement | 0.59 ± 0.20 | 0.62 ± 0.14 | 0.62 |
| ratio parent vessel^{†} | | | |
| Plaque vs. arterial | 0.99 ± 0.94 | −0.02 ± 0.48 | <0.001* |
| enhancement | | | |

*P < 0.05
^{†}Reported as mean SI.

TABLE 15
Univariate and multivariate logistic regression.

| Univariate logistic regression | | | | Multivariate analysis | | |
|---|---|---|---|---|---|---|
| | | 95% | | | 95% | |
| Characteristic | OR* | CI | p-value | OR* | CI | p-value |
| Mean | 2.3 | 1.4-4.1 | 0.002 | | | |
| enhancement | | | | | | |
| ratio plaque | | | | | | |
| Plaque | 1.9 | 1.2-3.2 | 0.013 | | | |
| enhancement | | | | | | |
| variance | | | | | | |
| Plaque vs vessel | 7.4 | 3.2-22 | <0.001* | | | |
| enhancement | | | | | | |
| Location^{‡} | 1.5 | 0.6-4 | 0.4 | | | |
| Morphology | 10.1 | 3.7-30 | <0.001^{†} | | | |
| Area of stenosis | 4 | 2.3-8 | <0.001^{†} | | | |
| Stenosis | 2.4 | 1.5-4.3 | 0.001^{†} | 1.3 | 0.7-2.7 | 0.4 |
| Gd-Uptake | 3.9 | 2.1-8.3 | <0.001^{†} | 2.3 | 1.1-5.3 | 0.033^{†} |
| PB | 6.1 | 3-14.9 | <0.001^{†} | 3.7 | 1.6-10 | 0.004^{†} |

*One SD increase in each variable
^{†}P < 0.05
^{‡}Anterior vs. posterior circulation

What is claimed is:

1. A method for analyzing an arterial wall comprising:
obtaining a scan of a target area;
processing the scan;
analyzing surface of the target area;
calculating one or more metrics for comparison; and
outputting an instability outcome.

2. The method of claim 1, wherein the one or more metrics include one or more of diameter, size ratio, aspect ratio, ellipticity index, non-sphericity index, undulation index, surface area, volume, wall thickness, bleb percentage, and mural thrombosis.

3. The method of claim 1 or 2, wherein the instability outcome is one or more of a likelihood of stroke, indication of aneurysm size, likelihood of rupture, or indication of inflammation of an artery.

4. The method of any of claims 1-3, further comprising aligning the scan to a template.

5. The method of any of claims 1-4, further comprising extracting spoke variables.

6. The method of any of claims 1-5, further comprising extracting surface measures.

7. The method of any of claims 1-6, further comprising measuring gadolinium uptake.

8. The method of any of claims 1-7, further comprising determining one or more of aneurysm wall enhancement, generalized aneurysm wall enhancement, specific aneurysm wall enhancement, circumferential aneurysm wall enhancement, and focal aneurysm wall enhancement.

9. The method of any of claims 1-8, further comprising projecting spokes into the target area.

10. The method of claim 9, further comprising probing a signal intensity along each spoke.

11. The method of claim 10, further comprising store a maximum signal intensity for each spoke.

12. The method of any of claims 9-11, further comprising normalizing spoke values to a reference structure.

13. The method of claim 12, wherein the reference structure is a pituitary stalk or a corpus callosum.

14. The method of claim 13, wherein the reference structure is the corpus callosum.

15. The method of any of claims 1-14, further comprising calculating morphological indices.

16. The method of any of claims 1-15, wherein gadolinium is administered prior to the scan.

17. The method of any of claims 1-16, further comprising determining a contrast ratio.

18. The method of claim 17, wherein the contrast ratio is normalized to a reference structure.

19. The method of any of claims 1-18, further comprising comparing the one or more metrics for two scan, one taken before administration of contrast, and one taken after administration of contrast.
